(19) United States
(12) Patent Application Publication
(10) Pub. No.: US 2012/0053170 A1
(43) Pub. Date: Mar. 1, 2012

(54) NICOTINAMIDE DERIVATIVES, PREPARATION THEREOF, AND THERAPEUTIC USE THEREOF AS ANTICANCER DRUGS

(75) Inventors: Jérome Arigon, Paris (FR); Claude Bernhart, Paris (FR); Monsif Bouaboula, Paris (FR); Romain Combet, Paris (FR); Sandrine Hilairet, Paris (FR); Samir Jegham, Paris (FR)

(73) Assignee: SANOFI, Paris (FR)

(21) Appl. No.: 13/258,220

(22) PCT Filed: Mar. 22, 2010
(86) PCT No.: PCT/FR10/50511

§ 371 Date: Nov. 10, 2011

(30) Foreign Application Priority Data
Mar. 24, 2009 (FR) 0901365

(51) Int. Cl. A61K 31/55; C07D 417/14; C07D 401/12; C07F 5/02; C07F 5/04; C07D 239/42; C07D 213/82; C07D 239/30; A61K 31/4545; A61K 31/506; A61K 31/541; A61K 31/444; A61K 31/496; A61P 35/00; C07D 401/14

(52) U.S. Cl. 514/217.04; 546/194; 544/328; 544/58.2; 546/256; 546/262; 540/597; 544/364; 546/13; 558/288; 562/7; 544/329; 546/309; 544/334; 514/318; 514/256; 514/227.8; 514/333; 514/332; 514/253.13

(57) ABSTRACT

The present invention relates to nicotinamide derivatives of formula (I), to compositions containing same, and to the therapeutic use thereof, in particular as anticancer drugs. The invention also relates to the method for preparing said compounds, as well as to some of the intermediate products.

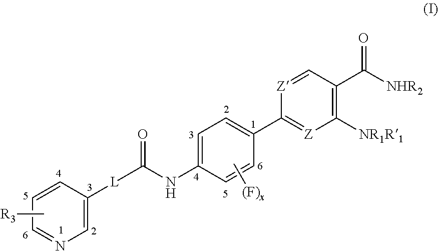

The present invention relates to nicotinamide derivatives, to the compositions comprising them and to their therapeutic application, in particular as anticancer drugs. The invention also relates to the process for the preparation of these compounds and to some of the intermediates.

### THE PRIOR ART

International Application WO 2005/051366 describes compounds of general formula (A):

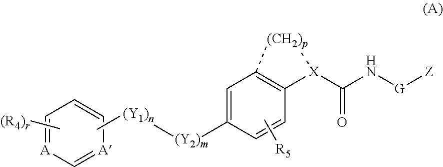

in which Z represents a phenyl or indanyl group and not a pyridinyl group.

International Application WO 2007/016538 describes compounds of general formula (B):

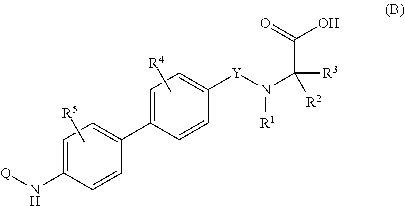

in which Q can represent an R₁₃—NR₁₂—C(═O)— group, it being possible for R₁₃ to be a 2-, 3- or 4-pyridinyl group, R₄ and R₅ representing a hydrogen atom or an alkyl, alkoxy, —OH, —CF₃ or —CN group. These compounds are used in the treatment of obesity.

### DESCRIPTION OF THE INVENTION

### Definitions Used

In the context of the present invention:
halogen atom is understood to mean: a fluorine, chlorine, bromine or iodine atom;
alkyl group is understood to mean; a saturated aliphatic hydrocarbon group comprising from 1 to 6 carbon atoms (advantageously from 1 to 4 carbon atoms) obtained by removing a hydrogen atom from an alkane. The alkyl group can be linear or branched. Mention may be made, by way of examples, of the methyl, ethyl, propyl, isopropyl, butyl, isobutyl, tert-butyl, pentyl, 2,2-dimethylpropyl or hexyl groups;
alkoxy group is understood to mean: an —O-alkyl group, where the alkyl group is as defined above;
cycloalkyl group is understood to mean: a cyclic alkyl group comprising between 3 and 8 carbon atoms, all the carbon atoms being involved in the cyclic structure. Mention may be made, by way of examples, of the cyclopropyl, cyclobutyl, cyclopentyl or cyclohexyl groups;
heterocycloalkyl group is understood to mean: a cycloalkyl group comprising at least one heteroatom (O, S, N) involved in the ring and connected to the carbon atoms forming the ring. Mention may be made, by way of examples, of the pyrrolidinyl, piperidinyl, piperazinyl or N—(C₁-C₄ alkyl)piperazinyl, azepanyl, thiomorpholinyl, 1-oxothiomorpholinyl or 1,1-dioxothiomorpholinyl groups.

According to a 1^{st} aspect, a subject-matter of the present invention is a compound of formula (I):

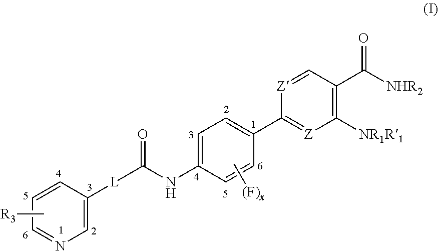

in which:
Z and Z′ represent N or CH;
x is an integer having the value 1 or 2, representing the number of fluorine atom(s) attached to the central phenyl nucleus;
L represents a —CH═CH— or —(CH₂)ₙNH— group in which the NH group is attached to the C═O and n is an integer having the value 0, 1 or 2;
R₁ represents a hydrogen atom or a (C₁-C₆)alkyl, (C₃-C₆)cycloalkyl or phenyl group;
R′₁ represents a hydrogen atom or a (C₁-C₆)alkyl group;
R₂ represents:
   a (C₃-C₆)cycloalkyl group;
   a (C₁-C₆)alkyl group, optionally substituted by:
      one or more hydroxyl or (C₁-C₄)alkoxy groups;
      an —NRₐR_{b} group in which Rₐ and R_{b} represent, independently of one another, a hydrogen atom or a (C₁-C₆)alkyl group or form, together with the nitrogen atom to which they are connected, a (C₄-C₆)heterocycloalkyl group optionally comprising, in the ring, the —S(O)_{q}— group with q=0, 1 or 2 or the —NH— or —N(C₁-C₄ alkyl)- group and being optionally substituted by one or more substituent(s), which are identical to or different from one another when there are several of them, chosen from an —OH, (C₁-C₄)alkoxy or (C₁-C₄)alkyl group;
R₃ represents at least one substituent of the pyridine nucleus chosen from a hydrogen atom, a fluorine atom, a (C₁-C₄)alkyl group or an —NR_{c}R_{d} group in which R_{c} and R_{d} represent a hydrogen atom or a (C₁-C₄)alkyl group.

R₁ represents a hydrogen atom, a (C₁-C₆)alkyl group, a (C₃-C₆)cycloalkyl group, for example a cyclopropyl group, or a phenyl group. R′₁ represents a hydrogen atom or a (C₁-C₆)alkyl group. More particularly, R′₁ represents a hydrogen atom. R₁ and/or R′₁ can be chosen from those described in Table I.

R₂ represents:
a (C₃-C₆)cycloalkyl group, such as, for example, the cyclopropyl or cyclopentyl group;
a (C₁-C₆)alkyl group, optionally substituted by:
   one or more —OH or (C₁-C₄)alkoxy, for example methoxy, group(s);
   an —NRₐR_{b} group in which Rₐ and R_{b} represent, independently of one another, a hydrogen atom or a (C₁-C₆)alkyl group or form, together with the nitrogen atom to which they are connected, a (C₄-C₆)heterocycloalkyl group optionally comprising, in the ring, the —S(O)_{q}— group with q=0, 1 or 2 or the —NH— or —N(C₁-C₄ alkyl)- group. Preferably, q=1 or 2.

The heterocycloalkyl group formed by Rₐ and R_{b} can, for example, be the pyrrolidinyl

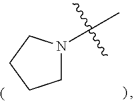

piperidinyl

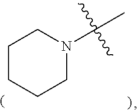

piperazinyl

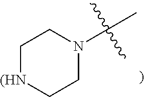

or N—(C₁-C₄ alkyl)piperazinyl

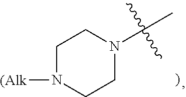

in particular N-methylpiperazinyl, azepanyl

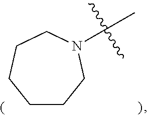

thiomorpholinyl

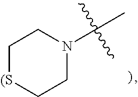

1-oxothiomorpholinyl

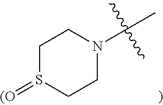

or 1,1-dioxothiomorpholinyl

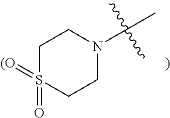

group.

The heterocycloalkyl group formed by Rₐ and R_{b} can optionally be substituted by one or more substituent(s), identical to or different from one another when there are several of them, chosen from: —OH; (C₁-C₄)alkoxy: for example methoxy; or (C₁-C₄)alkyl: for example methyl.

Thus, the substituted heterocycloalkyl can be the 3-hydroxypiperidinyl

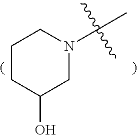

or 4-hydroxypiperidinyl

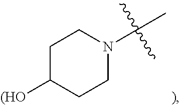

4-methoxypiperidinyl

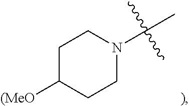

cis-3,5-dimethylpiperidinyl

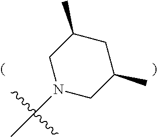

or cis-2,6-dimethylpiperidinyl

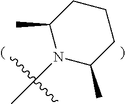

group.

R₂ can be chosen from one of those described in Table I.

The pyridine nucleus can comprise from 1 to 4 R₃ substituents chosen from a hydrogen atom, a fluorine atom, a (C₁-C₄)alkyl group or an —NR_{c}R_{d} group in which R_{c} and R_{d} represent a hydrogen atom or a (C₁-C₄)alkyl group. R₃ can be chosen from those described in Table I. Preferably, R₃ is in the 5 and/or 6 position on the pyridine nucleus. Preferably, the number of R₃ substituents is equal to 1 and/or R₃ is in the 5 or 6 position on the pyridine nucleus, as is represented below:

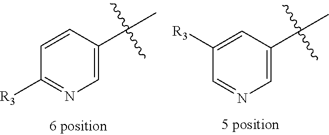

R₃ is more preferably still in the 6 position. Preferably. R₃ represents a hydrogen atom or —NH₂.

L represents a —CH═CH— or —(CH₂)ₙNH— group in which the NH group is attached to the C═O and n is an integer having the value 0, 1 or 2. Preferably, n is equal to 1. L can be one of those described in Table I. Preference is also given, in the case where L represents the —CH═CH— group, to the E isomers rather than the Z isomers.

Z and Z′ represent N or CH. For example, Z and Z′ can respectively represent N and CH, CH and CH or N and N:

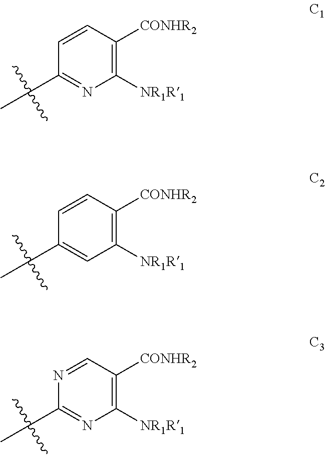

x is an integer having the value 1 or 2, representing the number of fluorine atom(s) attached to the central phenyl nucleus. More particularly, x has the value 1.

The subgroup of formula (I′):

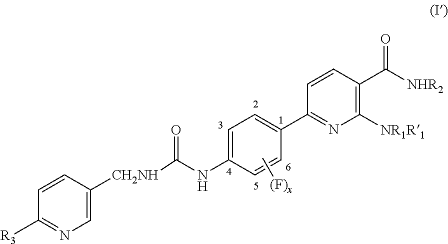

in which R₁, R′₁, R₂, R₃ and x are as defined above, is singled out.

The subgroup of formula (I″):

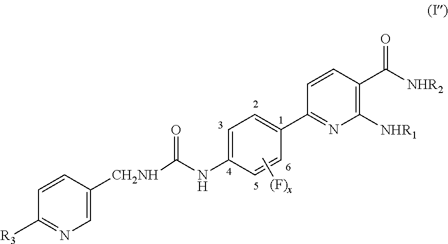

in which R₁ represents a (C₁-C₄)alkyl group, R₂ represents a (C₁-C₆)alkyl group optionally substituted by the —NRₐR_{b} group in which Rₐ and R_{b} form, together with the nitrogen atom to which they are connected, the (C₄-C₆)heterocycloalkyl group optionally comprising, in the ring, the —S(O)_{q}— group with q=0, 1 or 2 or the —NH— or —N(C₁-C₄ alkyl)- group and R₃ and x are as defined above, is singled out. More particularly, x has the value 1. More particularly still, x has the value 1 and the fluorine atom is in the 3 position.

Mention may be made, among the compounds which are subject-matters of the invention, of those in Table I.

The compounds of the invention, including the compounds given in the examples, can exist in the form of bases or of addition salts with acids. Such addition salts also come within the invention. These salts are advantageously prepared with pharmaceutically acceptable acids but the salts of other acids, for example of use in the purification or isolation of the compounds, also come within the invention. The compounds according to the invention can also exist in the form of hydrates or solvates, namely in the form of combinations or associations with one or more molecules of water or with a solvent. Such hydrates and solvates also come within the invention.

The compounds can comprise one or more asymmetric carbon atoms. They can thus exist in the form of enantiomers or diastereoisomers. These enantiomers and diastereoisomers, and their mixtures, come within the invention.

According to the present invention, the N-oxides of the compounds comprising an amine or a nitrogen atom also come within the invention.

According to a 2^{nd} aspect, a subject-matter of the invention is the process for the preparation of the compounds of the invention and some of the reaction intermediates.

Preparation of the Compounds of Formula (I) or (I′) for which L=—(CH₂)ₙNH—

These compounds can be prepared according to one of the following schemes 1-3.

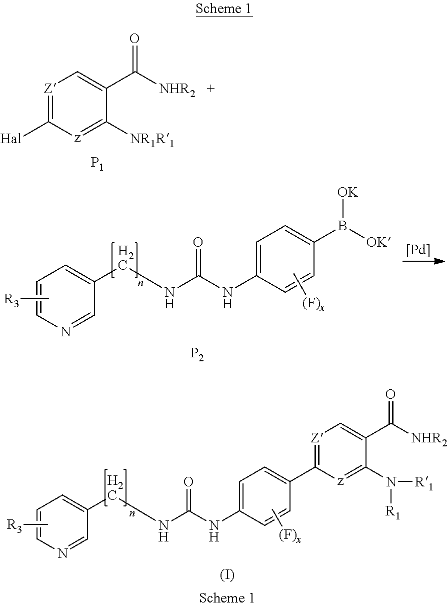

A coupling of Suzuki type of P₁ and P₂ is carried out. Hal represents a halogen atom (chlorine, bromine, iodine). The coupling is carried out in the presence of a palladium (in the (0) or (II) oxidation state) complex in a basic medium. The complex can, for example, be Pd(PPh₃)₄, PdCl₂(PPh₃)₂, Pd(OAc), PdCl₂(dppf) or bis[di(tert-butyl)(4-dimethylaminophenyl)-phosphine]dichloropalladium(II). The most frequently used complexes are palladium(0) complexes. The base can, for example, be K₂CO₃, NaHCO₃, Et₃N, K₃PO₄, Ba(OH)₂, NaOH, KF, CsF, Cs₂CO₃, and the like. The coupling can be carried out in a mixture of an ethereal solvent and of an alcohol, for example a dimethoxyethane (DME)/ethanol mixture; it can also be a toluene/water mixture. The temperature is between 50 and 120° C. The reaction time can, in some cases, be lengthy (see Ex. 1.3.).

Further details on Suzuki coupling, on the operating conditions and on the palladium complexes which can be used will be found in: N. Miyaura and A. Suzuki, Chem. Rev. 1995, 95, 2457-2483; A. Suzuki in “Metal-catalyzed cross-coupling reactions”; Diederich. F. and Stang, P. J., Editors, Wiley-VCH; Weinhein, Germany, 1998, chap. 2, 49-97; Littke, A. and Fu, G., Angew. Chem. Int. Ed., 1999, 38, 3387-3388 and Chemler, S. R. Angew. Chem. Int. Ed., 2001, 40, 4544-4568.

K and K′ represent a hydrogen atom, an alkyl group or an aryl group which are optionally connected to one another to form, together with the boron atom and the two oxygen atoms, a 5- to 7-membered ring optionally substituted by at least one (C₁-C₄)alkyl group or to which is optionally fused, over two consecutive carbon atoms on the said ring, a phenyl group. For example, use may be made of one of the following groups:

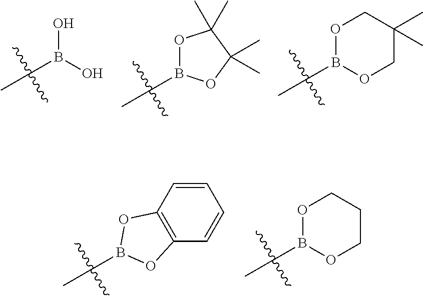

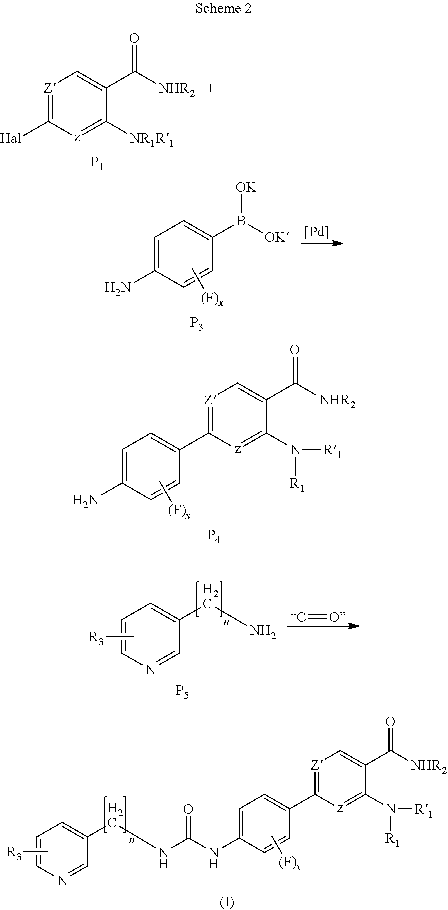

According to Scheme 2, a Suzuki coupling (see above) is carried out between P₁ and P₃, in order to obtain P₄, and then P₄ is reacted with P₅ in the presence of an agent which makes it possible to introduce the “C═O” unit (for example phosgene, triphosgene or N,N′-disuccinimidyl carbonate DSC). The reaction which makes it possible to introduce “C═O” is preferably carried out in the presence of a base, such as, for example, triethylannine, and at a temperature of between −5° C. and ambient temperature. The solvent can be THF. See Ex. 1.4.

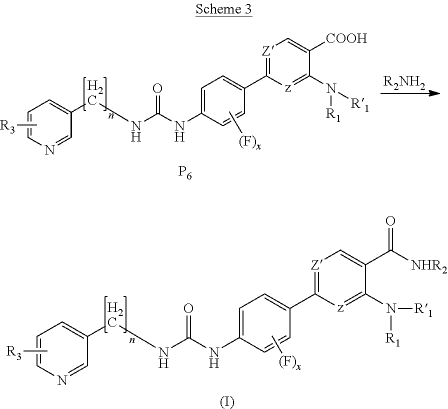

According to Scheme 3, the compound of formula (I) is obtained by an amidation reaction starting from P₆ and the amine R₂NH₂ or a salt of this amine, for example hydrochloride (see Ex. 3.2). The amidation can advantageously be carried out in the presence of an acid activator (also known as coupling agent), such as, for example, (benzotriazol-1-yloxy)tris(dimethylamino)phosphonium hexafluorophosphate (or BOP, CAS No. 56602-33-6, see also B. Castro and J. R. Dormoy, Tetrahedron Letters, 1975, 16, 1219). The reaction is preferably carried out in the presence of a base (such as triethylannine) at ambient temperature in a solvent, such as tetrahydrofuran (THE) or dimethylformamide (DMF).

P₆ is, for its part, obtained by a coupling reaction of Suzuki type between P₂ and the compound P₈ of formula:

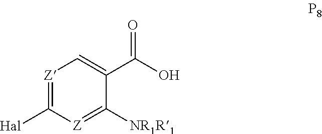

according to a scheme similar to Scheme 1.

### Preparation of P₁

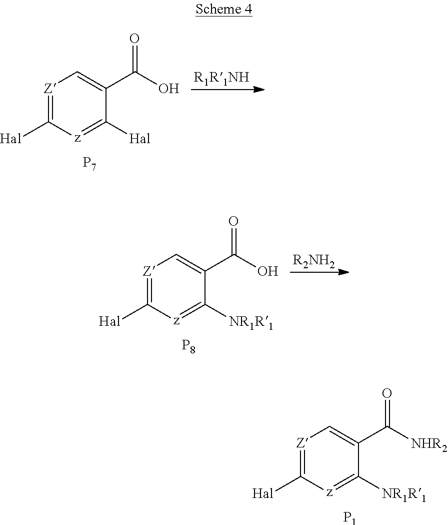

P₈ is obtained from the acid P₇ by monosubstitution by an amine of formula R₁R′₁NH. In the case of an aliphatic amine, the reaction can be carried out at ambient temperature and in a protic solvent, such as an alcohol or water, or in an aprotic solvent, such as THF. In the case of an aniline, a strong base, such as, for example, LiHMDS (((CH₃)₃Si)₂NLi), is added, and the reaction is carried out under hot conditions. The monosubstitution is described on pages 14 and 15 of FR 2917412, in the case where Z═N and Z′═CH, but can be applied to other Z/Z′ combinations. See also Ex. 1.1.
Z═N, Z′═CH: P₇ is a 2,6-dihalonicotinic acid, for example 2,6-dichloronicotinic acid, which is commercially available (see Ex. 1.1);
Z═N, Z′═N: P₇ is a 2,4-dihalopyrimidinecarboxylic acid, for example 2,4-dichloropyrimidinecarboxylic acid, which is commercially available (CAS No. 37131-89-8);
Z═CH, Z′═CH: P₇ is a 2,4-dihalobenzoic acid, for example 2,4-dichlorobenzoic acid, which is commercially available (CAS No. 50-84-0).

In the case where Z and Z′ both represent N and Hal represents a chlorine atom. P₈ can also be obtained from the commercial compound 2,4-dichloro-5-pyrimidinecarboxylic acid ethyl ester:

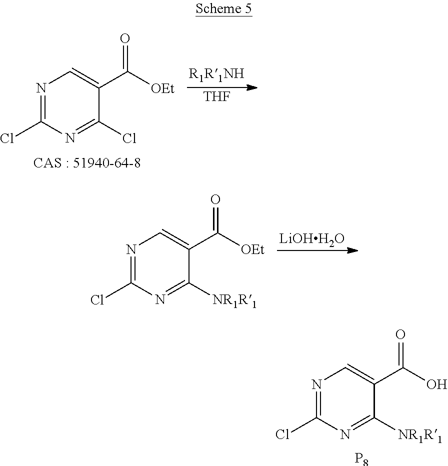

Scheme 5, which uses an ester functional group subsequently converted to an acid functional group, also applies to the case where Z═N and Z′═CH: see the conditions in Chem. Pharm. Bull., 2000, 48(12), 1847-1853 (reactions of Tables 1 and 2).

P₁ is obtained from the acid P₈ by amidation using the amine R₂NH₂ or a salt of this amine, for example the hydrochloride. The amidation can advantageously be carried out in the presence of an acid activator (also known as coupling agent), such as, for example, (benzotriazol-1-yloxy)tris(dimethylamino)phosphonium hexafluorophosphate (or BOP, CAS No. 56602-33-6, see also Castro. B. and Dormoy, J. R. Tetrahedron Letters, 1975, 16, 1219). The reaction is preferably carried out in the presence of a base (such as triethylamine) at ambient temperature in a solvent, such as tetrahydrofuran (THF) or dimethylformamide (DMF). See Ex. 1.2.

### Preparation of P₃

The compounds P₃ for which K and K′ form the following group

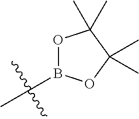

are commercially available or can be prepared according to the coupling reaction between a fluorinated bromoaniline and bis(pinacolato)diboron which is described in Scheme 2 on pages 150-151 of WO 2007/064931: 3-F(4-amino-3-fluorophenylboronic acid pinacol ester, CAS No. 819058-34-9, Boron Molecular Inc., PO Box 12592, Research Triangle Park, NC 27709); 2-F (4-amino-2-fluorophenylboronic acid pinacol ester, CAS No. 819057-45-9, Boron Molecular, described on page 185 of WO 2007/064931); 2-F, 5-F (CAS No. 939807-75-7, compound described on page 184 of WO 2007/064931); 3-F, 5-F (CAS No. 939968-08-8, described on page 182 of WO 2007/064931).

The compounds P₃ for which K and K′ represent a hydrogen atom can be prepared from the fluorinated bromoaniline by the reactions described in Tetrahedron Letters, 2003, 44, 7719-7722.

### Preparation of P₂

The compounds P₂ are obtained from the compounds P₃ and P₅ in the presence of an agent which makes it possible to introduce the “C═O” unit, according to a reaction as described above.

### Compounds R₂NH₂

The amines R₂NH₂ are commercial products or products already described in published documents; for example:
1-(2-aminoethyl)piperidine: CAS No. 27578-60-5, described in Justus Liebigs Annalen der Chemie, 1950, 566, 210-44, sold by ACROS;
1-piperidinepropanamine: CAS No. 3529-08-6, described in Bioorganic & Med. Chem. Lett., 2006, 16(7), 1938-1940;
1-piperidinebutanamine: CAS No. 74247-30-6, described in Bioorganic & Med. Chem. Lett., 2006, 16(7), 1938-1940;
1-(2-aminoethyl)-4-piperidinol: CAS No. 129999-60-6, described in J. Med. Chem., 2005, 48(21), 6690-6695, and on page 17 of WO 2005/061453 (ref. Ex. 10);
1-(2-aminoethyl)-3-piperidinol: CAS No. 847499-95-0, described in J. Med. Chem., 2005, 48(21), 6690-6695, and on page 16 of WO 2005/061453 (ref. Ex. 8);
2-(4-methoxy-1-piperidinyl)ethylamine: CAS No. 911300-69-1, described in J. Med. Chem., 2007, 50(20), 4818-4831;
pyrrolidineethanamine: CAS No. 7154-73-6, described in Anales de Quimica, 1974, 70(9-10), 733-737, sold by International Laboratory Ltd, 1067 Sneath Ln, San Bruno, Calif. 94066, USA;
1-piperazineethanamine: CAS No. 140-31-8, described in EP 151232;
azepan-1-ylethylamine: CAS No. 51388-00-2, described in Anales de Quimica, 1974, 70(9-10), 733-737;
2-(1,1-dioxothiomorpholin-4-yl)ethylamine: CAS No. 89937-52-0, sold by Intern. Lab. Ltd;
N-(2-aminoethyl)thiamorpholine 1-oxide: CAS No. 1017791-77-3, sold by Sinova Inc., 3 Bethesda Metro Center, Suite 700, Bethesda, Md., 20814, USA.

A method for producing compounds in which R₂ represents a (C₁-C₆)alkyl group substituted by the —NRₐR_{b} group in which Rₐ and R_{b} form, together with the nitrogen atom to which they are connected, the (C₄-C₆)heterocycloalkyl group optionally comprising, in the ring, the —S(O)_{q}— group with q=0, 1 or 2 or the —NH— or —N(C₁-C₄ alkyl)- group is described in Scheme 6, which takes its inspiration from Scheme 3 of Bioorg. Med. Chem., 2007, 15, 365-373 or from Scheme 2 of Bioorg. Med. Chem. Lett., 2008, 18, 1378-1381:

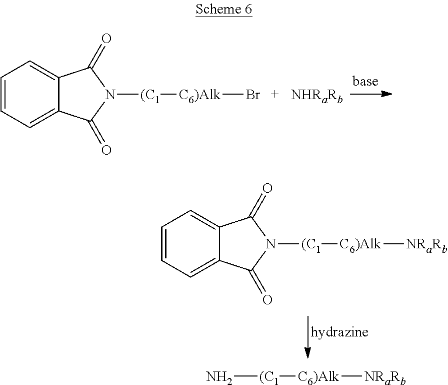

Another method, described in Scheme 6′, takes its inspiration from FIG. 2 of Bioorg. Med. Chem. Lett., 2006, 16, 1938-1940:

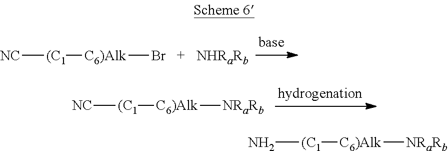

### Compounds P₅

P₅ can be either commercially available or prepared according to the methods known to a person skilled in the art. Use may be made, for example, of the hydrogenation of the cyano compound in order to obtain P₅ with n=1:

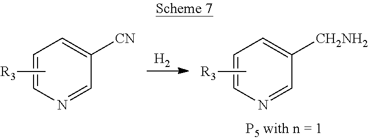

The hydrogenation conditions may be those described in Ex. 19 and 20 of WO 00/46179 or in Synlett, 2001, 10, 1623-1625.

The compounds 3-picolylamine (CAS No. 3731-52-0), 3-(2-aminoethyl)pyridine (CAS No. 20173-24-4), 2-amino-5-aminomethylpyridine (CAS No. 156973-09-0), 2-methyl-5-aminomethylpyridine (CAS No. 56622-54-9), 3-methyl-5-aminomethylpyridine (CAS No. 771574-45-9), 2-(BOC-amino)-5-(aminomethyl)pyridine (CAS No. 187237-37-2) and 2,5-diaminopyridine (CAS No. 4318-76-7) are commercial products. 2-Amino-5-aminomethylpyridine can also be prepared according to EP 0607804. 5-Aminomethyl-2-(dimethylamino)pyridine (CAS No. 354824-17-2) is commercially available or can be prepared according to J. Agr. Food Chem., 2008, 56(1), 204-212. 2-Amino-3-methyl-5-aminomethylpyridine (CAS No. 187163-76-4) can be obtained by catalytic hydrogenation of the compound 6-amino-5-methylnicotinonitrile (CAS No. 183428-91-3), the amine functional group being doubly protected by BOC. The catalytic hydrogenation of 6-methylamino-3-pyridinecarbonitrile (CAS No. 261715-36-0) makes possible access to 2-methylamino-5-aminomethylpyridine.

The preparation of 5-aminomethyl-2-(dimethylamino)pyridine (CAS No. 779324-37-7) and of 5-aminomethyl-2-(dimethylamino)pyridine (CAS No. 354824-17-2) in the hydrochloride form is also described on page 106 of WO 2007/044449 (Ex. 207 and 208).

Preparation of the Compounds of Formula (I) in which L=—CH═CH—

These compounds are obtained by an amidation reaction between P₄ and the acid P₁₀ or the acyl halide P′₁₀ derived from P₁₀. The amidation using P₁₀ can advantageously be carried out in the presence of an acid activator, such as, for example, BOP.

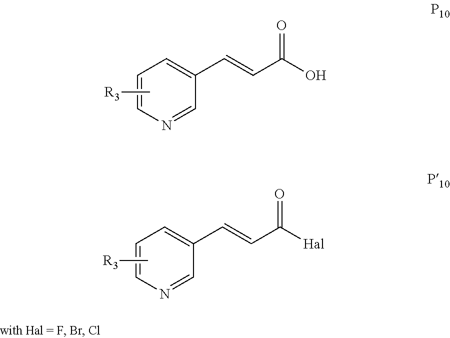

P₁₀ can be either commercially available or prepared according to the methods known to a person skilled in the art. For example, trans-3-(3-pyridyl)acrylic acid is sold by Sigma-Aldrich. (6-Aminopyridin-3-yl)acrylic acid (CAS No. 234098-57-8; compound E: CAS No. 167837-43-6) is described in J. Med. Chem., 2002, 45(15), 3246-3256 (see Scheme 4). P₁₀ can be prepared from a bromoaniline and acrylic acid according to the teaching of J. Med. Chem., 2002, 45(15), 3246-3256. Use may also be made of a coupling using a bromoaniline and an alkyl acrylate and then the ester functional group can be saponified to give the acid functional group (see, in this connection, the method which makes it possible to prepare (6-aminopyridin-3-yl)acrylic acid described in section [483] of US 2008269220 or [354] of EP1726580).

P₁₀ can also be prepared according to J. Org. Chem., 1998, 63, 8785-8789, from the corresponding β-formylpyridine or else according to J. Med. Chem., 1989, 32(3), 583-93 from 2-chloro-5-nitropyridine. The acyl halide P′₁₀ is obtained by a reaction known to a person skilled in the art from the acid P₁₀ and an acylating agent, such as, for example, SOCl₂ or (COCl)₂.

These compounds can also be prepared according to the following scheme 8:

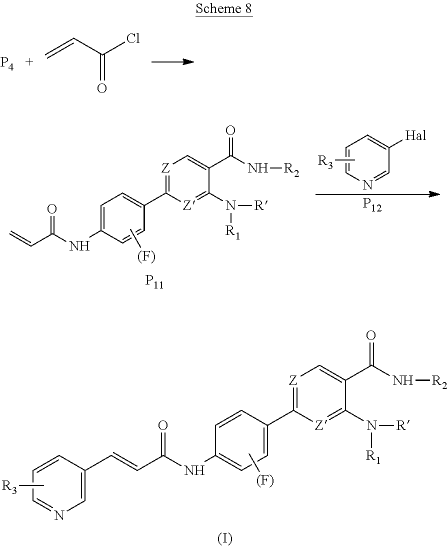

According to Scheme 8, P₄ is reacted with acryloyl chloride in the presence of a base, such as, for example, triethylamine, and at a temperature of between 0° C. and ambient temperature, in order to produce P₁₁. The solvent can be dichloromethane (DCM) (see Ex. 4.1).

P₁₁ is then reacted with P₁₂ (Hal represents a halogen atom) in the presence of a palladium complex, such as, for example, Pd(OAc)₂, tri(ortho-tolyl)phosphine and a base, such as, for example, diisopropylethylamine. The solvent can, for example, be propionitrile. The temperature is between ambient temperature and the reflux temperature of the solvent.

### Protection of the Primary or Secondary Amine Functional Group

It may be necessary to use, in at least one of the stages, a protective group (PG) in order to protect one or more chemical functional group(s), in particular a primary or secondary amine functional group. For example, when Rₐ and R_{b} both represent a hydrogen atom, the amidation of Scheme 3 is carried out using, for R₂NH₂, the compound ₂HN—(C₁-C₆)alkyl-NH-PG, where PG advantageously represents BOC (tert-butoxycarbonyl). Likewise, when the heterocycloalkyl group formed by Rₐ and R_{b} represents the piperazinyl

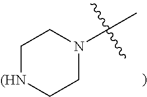

group, the —NH— functional group thereof can advantageously be protected using the following compound R₂NH₂

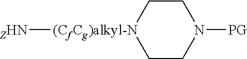

where PG advantageously represents BOC. Likewise, when R₃ represents the —NH₂ or —NHR_{c} group, the amine functional group can advantageously be protected by one or two PG group(s), preferably BOC or FMOC (9-fluorenylmethyl carbamate). Use may be made, for example, of the following compound P₅:

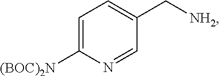

or else of the following compounds P₁₀ or P′₁₀:

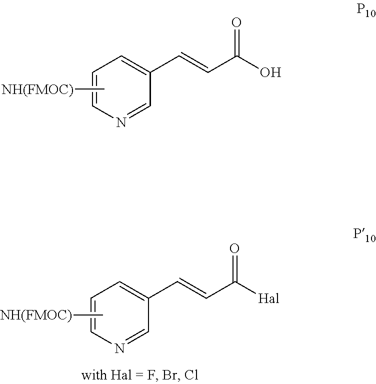

The chemical functional group(s) is/are subsequently obtained by a stage of deprotection (final or intermediate), the conditions of which depend on the nature of the functional group(s) protected and on the protective group used. Reference may be made to “Protective Groups in Organic Synthesis” by T. Greene, Wiley, 4^{th} ed., ISBN=978-0-471-69754-1, in particular to chap. 7 as regards the protective groups for the amine functional group. In the case of the protection of the —NH₂ or —NH— functional groups by BOC, the deprotection stage is carried out in an acidic medium using, for example. HCl or trifluoroacetic acid (TFA). Thus, if appropriate, the associated salt (hydrochloride or trifluoroacetate) is obtained.

### Preparation of the Salts

The salts are obtained during the deprotection stage described above or else by bringing the acid into contact with the compound in its base form.

In the preceding Schemes, the starting compounds and the reactants, when their method of preparation is not described, are commercially available or are described in the literature or else can be prepared according to methods which are described therein or which are known to a person skilled in the art. A person skilled in the art may also take his inspiration from the operating conditions given in the examples which are described below.

### Preparation of the N-Oxides

The N-oxides of the compounds comprising an amine or a nitrogen atom are prepared according to the methods known to a person skilled in the art by reaction of the amine with organic peracids, such as peracetic acid, trifluoroperacetic acid, performic acid, perbenzoic acid or the derivatives of perbenzoic acid, such as 3-chloroperbenzoic acid, at temperatures of between 0° C. and 90° C., preferably at temperatures of less than 50° C.

According to a 3^{rd} aspect, the invention relates to a pharmaceutical composition comprising a compound as defined above in combination with a pharmaceutically acceptable excipient. The excipient is chosen from the normal excipients known to a person skilled in the art according to the pharmaceutical form and the method of administration desired. The method of administration can, for example, be orally or intravenously.

According to a 4^{th} aspect, a subject-matter of the invention is a medicament which comprises a compound as defined above and the use of a compound as defined above in the manufacture of a medicament. It may be of use in treating a pathological condition, in particular cancer. The medicament (and a compound according to the invention) can be administered in combination with one (or more) anticancer drug(s). This treatment can be administered simultaneously, separately or else sequentially. The treatment will be adjusted by the practitioner according to the patient and the tumour to be treated.

According to a 5^{th} aspect, the invention also relates to a method for the treatment of the pathologies indicated above which comprises the administration, to a patient, of an effective dose of a compound according to the invention or one of its salts, the salts being pharmaceutically acceptable, or hydrates or solvates.

### EXAMPLES

The following examples illustrate the preparation of some compounds in accordance with the invention. The numbers of the compounds given in the examples refer to those given in the table below, in which the chemical structures and the physical properties of a few compounds according to the invention are illustrated.

In the examples, the following abbreviations are used:

AcOEt: ethyl acetate
MeOH: methanol
DIPEA: diisopropylethylamine

The compounds were analysed by coupled HPLC-UV-MS (liquid chromatography, ultraviolet (UV) detection and mass detection). The device used is composed of an Agilent chromatographic sequence equipped with an Agilent diode array detector and with a Waters ZQ single quadrupole mass spectrometer or a Waters Quattro-Micro triple quadrupole mass spectrometer.

The compounds were analysed by coupled HPLC-UV-MS (liquid chromatography, ultraviolet (UV) detection and mass detection). The device used is composed of a chromatographic sequence equipped with a diode array detector (Agilent HP1110 or Waters Acquity HPLC) and with a quadrupole mass spectrometer (Waters ZQ, QM or SQD).

### Mass Spectrometry Conditions

The liquid phase chromatography/mass spectrometer (LC/MS) spectra were recorded in positive electrospray (ESI) mode, in order to observe the ions resulting from the protonation of compounds analysed (MH⁺) or from the formation of adducts with other cations, such as Na⁺, K⁺, and the like. The HPLC conditions are chosen from one of the following methods:

| Conditions | TFA15 | TFA3 | TFA6 |
|---|---|---|---|
| Column | Symmetry C18 (50 × 2.1 | Acquity BEH C18 (50 × 2.1 mm; | Acquity BEH C18 (50 × 2.1 mm; |
| | mm; 3.5 μm) | 1.7 μm) | 1.7 μm) |
| Eluent A | H₂O + TFA 0.005% at | H₂O + TFA 0.05% at | H₂O + TFA 0.05% at |
| | approximately pH 3.1 | approximately pH 3.1/CH₃CN | approximately pH 3.1/CH₃CN |
| | | (97/3) | (97/3) |
| Eluent B | CH₃CN + TFA 0.005% | CH₃CN + TFA 0.005% | CH₃CN + TFA 0.005% |
| Gradient | 100:0 (0 min) [symbol] 10:90 | 100:0 or 99:1 (0 min) [symbol] 5.95 | 100:0 or 99:1 (0 min) [symbol] 5.95 (4.8 |
| A:B | (10 min) [symbol] 10:90 (15 | (2.3 min) [symbol] 5:95 (2.9 min) [symbol] | min) [symbol] 5:95 (6 min) [symbol] 100:0 or |
| | min) [symbol] 100:0 (16 min) | 100:0 or 99:1 (3 min) [symbol] 100:0 | 99:1 (6.1 min) [symbol] 100:0 or 99:1 |
| | [symbol] 100:0 (20 min) | or 99:1 (3.5 min) | (6.6 min) |
| T. column | 30° C. | 40° C. | 40° C. |
| Flow rate | 0.4 ml/min | 1 ml/min | 1 ml/min |
| Detection | λ = 220 nm | λ = 220 nm | λ = 220 nm |

### NMR Conditions

The ¹H NMR spectra are recorded on a Bruker Avance 250/Bruker Avance 400 or Bruker Avance II 500 spectrometer. The central peak of the d₆-DMSO (2.50 ppm) is used as internal reference. The following abbreviations are used: s: singlet; d: doublet; dd: split doublet; t: triplet; q; quartet; m: broad unresolved peak/multiplet; br.s: broad signal.

### Example 1

### 6-{4-[3-(6-aminopyridin-3-ylmethyl)ureido]-3-fluorophenyl}-2-ethylamino-N-[2-(piperidin-1-yl)ethyl]nicotinamide (compound No. 1 prepared according to Scheme 2)

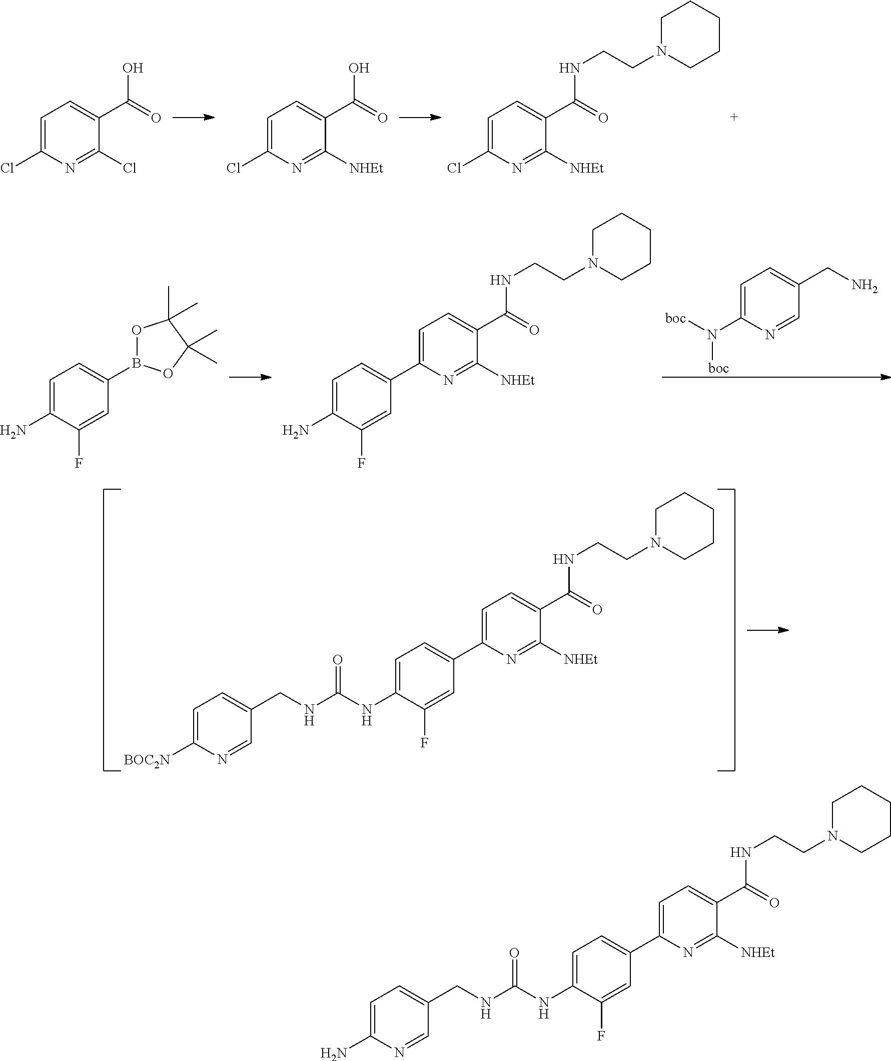

### 1.1. 6-Chloro-2-(ethylamino)nicotinic acid

26.1 g (0.136 mol) of 2,6-dichloronicotinic acid and 180 ml of 70% aqueous ethylamine solution are mixed in a round-bottom flask. The mixture is stirred at ambient temperature (AT) for 5 days. It is evaporated under reduced pressure (RP). The residue is taken up in 100 ml of water. The solution is cooled with an ice bath and acidified to pH 3 with a 5N HCl solution. The precipitate is filtered off, washed with cold water and dried under vacuum over P₂O₅ at 60° C. 24.93 g (91.4%) of white solid are obtained. M.p.=157-159° C.

### 1.2. 6-Chloro-2-ethylamino-N-[2-(piperidin-1-yl)ethyl]nicotinamide

5.0 g (24.92 mmol) of 6-chloro-2-(ethylamino)nicotinic acid are dissolved in 300 ml of THF in a round-bottom flask. 10.41 ml (74.77 mmol) of triethylamine, then 7.08 ml (49.84 mmol) of 1-(2-aminoethyl)piperidine and subsequently 11.02 g (24.92 mmol) of BOP are added. The mixture is stirred at AT for 15 h. The solvent is evaporated and the residue is taken up in ethyl acetate. The organic phase is washed with water and then with a saturated NaCl solution. It is dried over Na₂SO₄, filtered and evaporated. The residue is purified by flash chromatography (1 to 10% DCM-MeOH gradient). 7.5 g are obtained (yd: 96.8%). LCMS: M⁺ 310, rt (retention time)=1.01 min.

### 1.3. 6-(4-Amino-3-fluorophenyl)-2-ethylamino-N-[2-(piperidin-1-yl)ethyl]nicotinamide

5 g (16.1 mmol) of 6-chloro-2-ethylamino-N-[2-(piperidin-1-yl)ethyl]nicotinamide are introduced into a 1 litre three-necked flask. 4-Amino-3-fluorophenylboronic acid pinacol ester (1.1 eq., 4.2 g), 300 ml of 1,2-dimethoxyethane, 60 ml of ethanol and 120 ml of a saturated NaHCO₃ solution are added. Argon is bubbled in for 15 min and then palladiumtetrakis Pd(PPh)₄ (0.1 eq., 1.86 g) is added. The mixture is heated at reflux (˜100° C.) for 16 h. The mixture is concentrated, the residue is taken up in DCM and the organic phase is washed with H₂O, twice, and H₂O/NaCl, dried over sodium sulphate and concentrated. The product is subjected to flash chromatography on a column of silica, 400 g, 99/1 to 90/10 DCM/methanol gradient, 4.8 g (yd=78%) of 6-(4-amino-3-fluorophenyl)-2-ethylamino-N-[2-(piperidin-1-yl)ethyl]nicotinamide are obtained. LCMS (TFA3): MH⁺ 386, rt=0.90 min.

### 1.4. 6-{4-[3-(6-Aminopyridin-3-ylmethyl)ureido]-3-fluorophenyl}-2-ethylamino-N-[2-(piperidin-1-yl)ethyl]nicotinamide

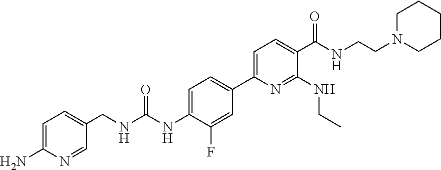

3.5 g (9.1 mmol) of 6-(4-amino-3-fluorophenyl)-2-ethylamino-N-[2-(piperidin-1-yl)ethyl]-nicotinamide are dissolved in 300 ml of anhydrous THF in a 1 litre round-bottom flask. DMAP (1.2 eq., 1.33 g) and N,N′-disuccinimidyl carbonate ([74124-79-1], 1.2 eq., 2.8 g) are added. The mixture is stirred at AT for 5 h. Triethylannine (3 eq., 3.8 ml) and 2-[di(boc)amino]-5-(aminomethyl)pyridine (1.2 eq., 3.53 g) are then added and the mixture is stirred overnight at AT. The mixture is concentrated. The residue is taken up in DCM and the organic phase is washed with H₂O, twice, and H₂O/NaCl, dried and concentrated. The residue is subjected to flash chromatography on silica, 95/5 to 79/20 DCM/MeOH gradient+1% of 20% NH₄OH. After concentrating, the fraction thus obtained is taken up in 200 ml of DCM and then 35 ml (50 eq.) of TFA are added under cold conditions. The mixture is stirred at AT until the “di(boc)amino” product has disappeared. The mixture is concentrated and then the residue is taken up in a 10% Na₂CO₃ solution. The organic phase is extracted with DCM and concentrated. The residue is crystallized from ethyl acetate under hot conditions. The product is filtered off, rinsed with AcOEt and dried in an oven. 3 g (yd=63%) of 6-{4-[3-(6-aminopyridin-3-ylmethyl)ureido]-3-fluorophenyl}-2-ethylamino-N-[2-(piperidin-1-yl)ethyl]nicotinamide are obtained, LCMS (TFA3): MH+ 535, rt=0.79 min; ¹H NMR (250 MHz, d₆-DMSO) δ ppm 1.22 (t, 3H), 1.29-1.68 (m, 6H), 2.26-2.47 (m, 6H), 3.28-3.42 (m, 2H), 3.43-3.62 (m, 2H), 4.13 (d, 2H), 5.83 (s, 2H), 6.44 (d, 1H), 6.97 (t, 1H), 7.16 (d, 1H), 7.35 (dd, 1H), 7.79-8.07 (m, 4H), 8.28 (t, 1H), 8.33-8.46 (m, 2H), 8.49 (s, 1H). M.p. (melting point)=175-177° C.

### Example 2

### 6-{4-[3-(6-Aminopyridin-3-ylmethyl)ureido]-3-fluorophenyl}-2-ethylamino-N-(2-hydroxyethyl)nicotinamide (compound No. 8 prepared according to Scheme 1)

### 2.1. 6-Chloro-2-(ethylamino)-N-(2-hydroxyethyl)nicotinamide

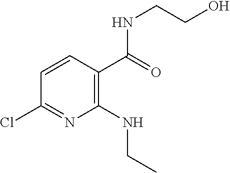

0.5 g (2.49 mmol) of 6-chloro-2-(ethylamino)nicotinic acid is dissolved in 30 ml of THF. 1.04 ml (0.76 mmol) of triethylamine, 0.304 g (4.98 mmol) of 2-hydroxyethylamine and 1.10 g (2.49 mmol) of BOP are added. The mixture is stirred at AT for 70 h. The solvent is evaporated and the residue is taken up in ethyl acetate; the organic phase is washed with water and then with a saturated NaCl solution. It is dried over Na₂SO₄, filtered and evaporated. The residue is purified by flash chromatography (DCM/MeOH 1-5%). 600 mg (yd=99%) are obtained. LCMS (TFA3): MH+ 244, rt=1.03 min.

### 2.2. Di(tert-butyl){5-[({[2-fluoro-4-(4,4,5,5-tetramethyl-1,3,2-dioxaborolan-2-yl)phenyl]carbamoyl}amino)methyl]pyridin-2-yl}imidodicarbonate

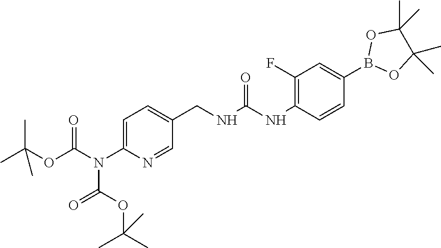

5.0 g (21.09 mmol) of 2-fluoro-4-(4,4,5,5-tetramethyl-1,3,2-dioxaborolan-2-yl)aniline and 3.09 g (25.31 mmol) of DMAP (4-dimethylaminopyridine) are dissolved in 500 ml of THF. 6.48 g (23.31 mmol) of DSC are added and the mixture is stirred at AT for 18 h. 8.81 ml (63.27 mmol) of triethylamine and 8.18 g (23.31 mmol) of di(tert-butyl) [5-(aminomethyl)pyridin-2-yl]imidodicarbonate are added. The mixture is stirred at AT for 5 h. The solvent is evaporated and the residue is taken up in DCM. The organic phase is washed with water and than with a saturated NaCl solution. It is dried over sodium sulphate, filtered and evaporated. The residue is purified by flash chromatography. 12 g of product composed of a 50/50 mixture of pinacolic ester and of boronic acid are obtained. LCMS (LS) MH+ 587, rt=6.17 min, and MH+ 505, rt=4.97 min.

### 2.3. Di(tert-butyl)[5-({[(4-{6-(ethylamino)-5-[(2-hydroxyethyl)carbamoyl]pyridin-2-yl}-2-fluorophenyl)carbamoyl]amino}methyl)pyridin-2-yl]imidodicarbonate

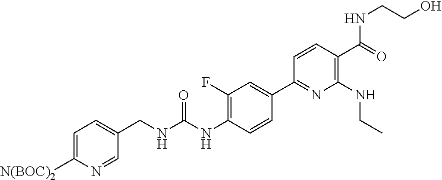

0.3 g (1.23 mmol) of compound obtained in stage 2.1, 0.794 g (1.35 mmol) of compound obtained in stage 2.2, 15 ml of saturated NaHCO₃ solution, 38 ml of DME and 7 ml of ethanol are placed in a three-necked flask. The mixture is degassed with argon and then 0.142 g (0.12 mmol) of Pd(PPh₃)₄ is added. The mixture is heated at reflux for 6 h. The solvents are evaporated and the residue is taken up in DCM. The organic phase is washed with water and then with a saturated NaCl solution. It is dried over Na₂SO₄, filtered and evaporated. The residue is purified by flash chromatography (DCM/MeOH 0-15%). 600 mg (yd=73%) are obtained. LCMS (TFA3): MH+ 668, rt=1.44 min.

### 2.4. 6-{4-[3-(6-Aminopyridin-3-ylmethyl)ureido]-3-fluorophenyl}-2-ethylamino-N-2-hydroxyethyl)nicotinamide

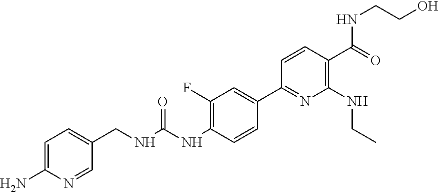

0.6 g (0.9 mmol) of the compound obtained in stage 2.3 is dissolved in 20 ml of DCM. The solution is cooled with an ice bath and 2.08 ml (27 mmol) of TFA are added. The mixture is stirred at AT for 18 h. The solvents are evaporated and the residue is taken up in an Na₂CO₃ solution. The product is filtered off, rinsed with water and dried in an oven over P₂O₅. 200 mg (yd=47.6%) are obtained. LCMS (TFA3): MH+ 468, rt=0.72 min; ¹H NMR (250 MHz, d₆-DMSO) δ ppm 1.22 (t, 3H), 331 (s, 2H), 3.43-3.62 (m, 4H), 4.13 (d, 2H), 4.71 (t, 1H), 5.83 (s, 2H), 6.44 (d, 1H), 6.97 (t, 1H), 7.16 (d, 1H), 7.35 (dd, 1H), 7.78-7.98 (m, 3H), 8.01 (d, 1H), 8.28 (t, 1H), 8.34-8.47 (m, 2H), 8.49 (d, 1H).

### Example 3

### 6-{4-[3-(6-Aminopyridin-3-ylmethyl)ureido]-3-fluorophenyl}-N-[2-(azepan-1-yl)ethyl]-2-(ethylamino)nicotinamide (compound No. 15 prepared according to Scheme 3)

### 3.1. 6-(4-{[({6-[Bis-tert-butoxycarbonyl)amino]pyridin-3-yl}methyl)carbamoyl]amino}-3-fluorophenyl)-2-(ethylamino)nicotinic acid

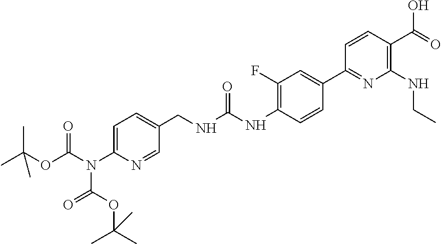

1.2 g (5.98 mmol) of 6-chloro-2-(ethylamino)nicotinic acid, 3.86 g (6.58 mmol) of di(tert-butyl) {5-[({[2-fluoro-4-(4,4,5,5-tetramethyl-1,3,2-dioxaborolan-2-yl)phenyl]carbamoyl}amino)methyl]-pyridin-2-yl}imidodicarbonate, 80 ml of DME, 15 ml of ethanol and 40 ml of saturated NaHCO₃ solution are placed in a three-necked flask. The mixture is degassed with argon and then heated at reflux for 18 h. The solvents are evaporated and the residue is taken up in water. The product is filtered off, rinsed with water and dried in an oven over P₂O₅. It is purified by flash chromatography. DCM/MeOH 1-20%. 1.8 g of a mixture of mono- and di(BOC) compounds are obtained. LCMS (LS) MH+ 525, rt=4.60 min, and MH+ 625, rt=5.59 min.

### 3.2. 6-{4-[3-(6-Aminopyridin-3-ylmethyl)ureido]-3-fluorophenyl}-N-[2-(azepan-1-yl)ethyl]-2-(ethylamino)nicotinamide

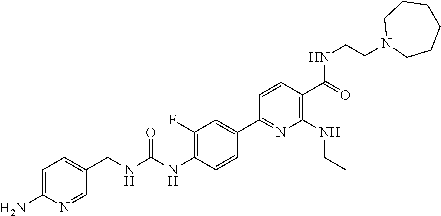

0.2 g (0.32 mmol) of the compound obtained in stage 3.1 is dissolved in 30 ml of THF. 0.115 g (0.64 mmol) of 2-(azepan-1-yl)ethanamine hydrochloride, 0.18 ml (0.13 mmol) of triethylannine and 0.142 g (0.32 mmol) of BOP are added. The mixture is stirred at AT for 18 h. It is evaporated, the residue is taken up in DCM and the organic phase is washed with water and then with a saturated NaCl solution. It is dried over Na₂SO₄, filtered and evaporated. The residue is purified by flash chromatography. DCM/MeOH 0-10%. 0.250 g of a mono- and di(BOC) mixture is obtained. This product is dissolved in 15 ml of DCM, the solution is cooled with an ice bath and 0.5 ml of TFA is added. The mixture is stirred at AT for 18 h. It is evaporated and the residue is taken up in an Na₂CO₃ solution. The precipitate is filtered off, washed with water and dried in an oven over P₂O₅. 0.13 g (yd=7496) is obtained. LCMS (TFA3) MH+ 549, rt=0.85 min; ¹H NMR (400 MHz, d₆-DMSO) [symbol] ppm 1.22 (t, 3H), 1.56 (m, 8H), 2.67 (m, 6H), 3.32 (m, 2H), 3.45-3.63 (m, 2H), 4.13 (d, 2H), 5.84 (s, 2H), 6.43 (d, 1H), 6.97 (t, 1H), 7.15 (d, 1H), 7.34 (d, 1H), 7.83-8.00 (m, 4H), 8.27 (t, 1H), 8.35 (t, 1 H), 8.41 (t, 1H), 8.49 (s, 1H).

### Example 4

### 6-{4-[(E)-3-(6-Aminopyridin-3-yl)acryloylamino]-3-fluorophenyl}-2-ethylamino-N-[2-(piperidin-1-yl)ethyl]nicotinamide (compound No. 43 prepared according to Scheme 8)

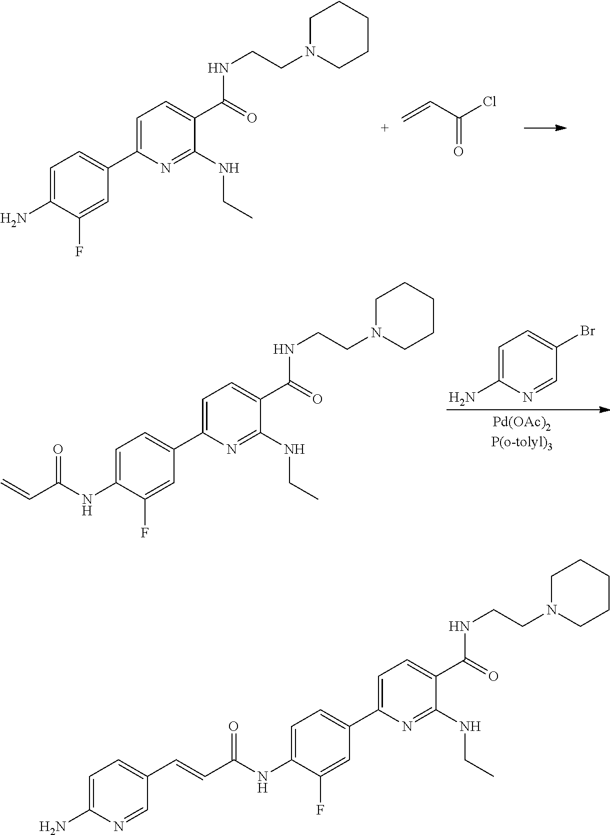

### 4.1. 6-(4-Acryloylamino-3-fluorophenyl)-2-ethylamino-N-[2-(piperidin-1-yl)ethyl]-nicotinamide

0.385 g (1 mmol) of the compound obtained in stage 1.3 is dissolved in 20 ml of DCM. 0.28 ml (2 mmol) of triethylamine and 0.111 g (1.1 mmol) of DMAP are added, followed by 0.2 ml (2.2 mmol) of acryloyl chloride. The mixture is stirred at ambient temperature for 18 h. The solvents are evaporated and the residue is taken up in DCM. The organic phase is washed with an Na₂CO₃ solution and then with a saturated NaCl solution. It is dried over sodium sulphate and filtered and then the filtrate is evaporated. The residue is purified by flash chromatography (95/5/0.2 DCM/CH₃OH/20% NH₄OH). 0.195 g (44.4%) is obtained. LCMS (TFA3) MH+ 440, rt=2.44 min.

### 4.2. 6-{4-[(E)-3-(6-Aminopyridin-3-yl)acryloylamino]-3-fluorophenyl}-2-ethylamino-N-[2-(piperidin-1-yl)ethyl]nicotinamide

0.187 g (0.43 mmol) of the compound obtained in stage 4.1 is dissolved in 15 ml of propionitrile. 0.074 g (0.43 mmol) of 2-amino-5-bromopyridine and 0.11 ml (0.64 mmol) of DIPEA are added. The mixture is degassed with argon for 30 minutes and then 0.01 g (0.04 mmol) of Pd(OAc)₂ and 0.022 g (0.07 mmol) of tri(ortho-tolyl)phosphine are added. The mixture is brought to reflux for 3 h. After returning to ambient temperature, the mixture is diluted with DCM and filtered through a Whatman filter. The filtrate is evaporated and the residue is purified by flash chromatography, 85/15/0.2 DCM/CH₃OH/20% NH₄OH. 0.120 g (53%) is obtained.

### Example 5

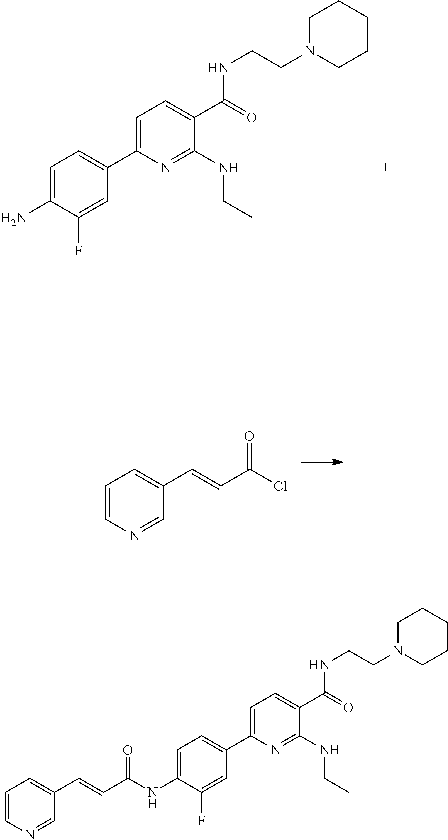

0.25 g (0.65 mmol) of the compound obtained in stage 1.3 is dissolved in 20 ml of DCM, and 0.27 ml (1.95 mmol) of triethylamine is added. The reaction medium is cooled in an ice bath and 0.163 g (0.97 mmol) of (E)-3-(pyridin-3-yl)acryloyl chloride is added dropwise. Stirring is carried out at ambient temperature for 18 h. The organic solution is washed with a 10% NaOH solution, dried over Na₂SO₄ and filtered and the filtrate is evaporated. The residue is purified by flash chromatography, SiO₂, C18, CH₃OH/H₂O 50/50-90/10. 0.035 g (10.4%) is obtained.

### Example 6

### 6-{4-[3-(6-Aminopyridin-3-ylmethyl)ureido]-3-fluorophenyl}-2-ethylamino-N-[2-(1-oxypiperidin-1-yl)ethyl]nicotinamide (compound No. 45)

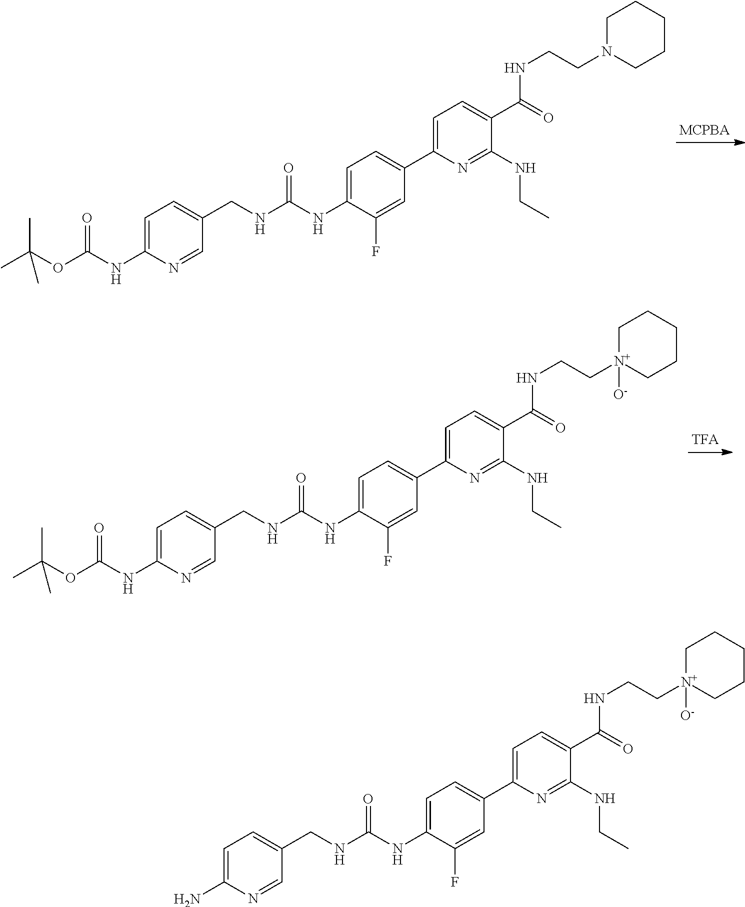

### 6.1. {5-[3-(4-{6-Ethylamino-5-[2-(1-oxypiperidin-1-yl)ethylcarbamoyl]pyridin-2-yl}-2-fluorophenyl)ureidomethyl]pyridin-2-yl}carbamic acid tert-butyl ester

0.35 g (0.55 mmol) of [5-(3-{4-[6-ethylamino-5-(2-(piperidin-1-yl)ethylcarbamoyl)pyridin-2-yl]-2-fluorophenyl}ureidomethyl)pyridin-2-yl]carbamic acid tert-butyl ester is suspended in 35 ml of DCM and 10 ml of CHCl₃. The suspension is cooled with an ice bath and 0.105 g (0.61 mmol) of meta-chloroperbenzoic acid is added. The mixture is stirred under cold conditions for 0.5 h and then at ambient temperature for 2 h. The organic phase is washed with an NaHCO₃ solution, then with H₂O and then with a saturated NaCl solution. It is dried over Na₂SO₄, filtered and evaporated. The residue is purified by flash chromatography on neutral Al₂O₃, DCM/CH₃OH— 98/2 to 92/8. 0.340 g (94.7%) is obtained. LCMS (TFA3) MH+ 651, rt=2.07 min.

### 6.2. 6-{4-[3-(6-Aminopyridin-3-ylmethyl)ureido]-3-fluorophenyl}-2-ethylamino-N-[2-(1-oxypiperidin-1-yl)ethyl]nicotinamide

0.328 g (0.5 mmol) of the compound obtained in stage 6.1 is dissolved in 20 ml of CH₂Cl₂. The solution is cooled with an ice bath and 0.85 ml (11.1 mmol) of TEA is added. The mixture is stirred at ambient temperature for 44 h. It is evaporated and the residue is taken up in a 10% Na₂CO₃ solution and extracted with DCM. The organic phase is dried over Na₂SO₄, filtered and evaporated. The residue is purified by flash chromatography on neutral Al₂O₃, DCM/CH₃OH-95/5 to 88/12. 0.213 g (76.9%) is obtained.

### ¹H NMR of the Compounds in Table 1

The chemical shifts 8 are given in ppm.

Compound No. 2: (400 MHz) 1.22 (t, 3H), 1.35-1.81 (m, 6H), 2.54-3.16 (m, 6H), 3.45-3.57 (m, 4H), 4.13 (d, 2H), 5.88 (s, 2H), 6.44 (d, 1H), 7.02-7.14 (m, 2H), 7.35 (dd, 1H), 7.85-7.92 (m, 2H), 7.98 (d, 1H), 8.16 (dd, 1H), 8.35 (t, 1H), 8.61 (br. s., 1H), 8.75 (s, 1H).

Compound No. 3: (250 MHz) 1.22 (t, 3H), 1.32-1.60 (m, 6H), 2.40-2.06 (m, 6H), 3.29-3.46 (m, 2H), 3.58 (m, 2H), 4.13 (d, 2H), 5.84 (s, 2H), 6.44 (d, 1H), 7.03 (t, 1H), 7.35 (dd, 1H), 7.88 (d, 1H), 8.00-8.19 (m, 2H), 8.25-8.39 (m, 1H), 8.50-8.63 (m, 2H), 8.68 (s, 1H), 8.75 (t, 1 H).

Compound No. 4: (250 MHz) 1.22 (t, 3H), 1.32-1.60 (m, 6H), 2.40-2.65 (m, 6H), 3.29-3.46 (m, 2H), 3.58 (m, 2H), 4.13 (d, 2H), 5.84 (s, 2H), 6.44 (d, 1H), 7.03 (t, 1H), 7.35 (dd, 1H), 7.88 (d, 1H), 8.00-8.19 (m, 2H), 8.25-8.39 (m, 1H), 8.50-8.63 (m, 2H), 8.68 (s, 1H), 8.75 (t, 1H).

Compound No. 5: (250 MHz) 1.28-1.65 (m, 6H), 2.32-2.47 (m, 6H), 3.35-3.52 (m, 2H), 4.13 (d, 2H), 5.84 (s, 2H), 6.44 (d, 1H), 7.02 (t, 2H), 7.26-7.52 (m, 4H), 7.75 (d, 2H), 7.82-8.04 (m, 3H), 8.16 (d, 1H), 8.34 (t, 1H), 8.53 (s, 1H), 8.69 (t, 1H), 11.06 (s, 1H).

Compound No. 6: (250 MHz) 0.97 (d, 6H), 1.21 (t, 3H), 1.57 (br. s., 1H), 2.58-2.87 (m, 3H), 3.23-3.34 (m, 2H), 3.43-3.61 (m, 2H), 4.13 (d, 2H), 5.84 (s, 2H), 6.44 (d, 1H), 6.99 (t, 1H), 7.16 (d, 1H), 7.35 (dd, 1H), 7.80-8.10 (m, 4H), 8.28 (t, 1H), 8.34-8.48 (m, 2H), 8.51 (d, 1H).

Compound No. 7: (250 MHz) 1.22 (t, 3H), 2.66 (t, 2H), 2.89-3.17 (m, 8H), 3.31-3.42 (m, 2H), 3.45-3.61 (m, 2H), 4.13 (d, 2H), 5.84 (s, 2H), 6.44 (d, 1H), 6.98 (t, 1H), 7.17 (d, 1H), 7.35 (dd, 1H), 7.83-8.03 (m, 4H), 8.28 (t, 1H), 8.34-8.46 (m, 2H), 8.50 (d, 1H).

Compound No. 9: (250 MHz) 0.49-0.60 (m, 2H), 0.77-0.94 (m, 2H), 1.29-1.57 (m, 6H), 2.28-2.48 (m, 6H), 2.99-3.11 (m, 1H), 3.31-3.42 (m, 2H), 4.13 (d, 2H), 5.83 (s, 2H), 6.44 (d, 1H), 7.03 (t, 1H), 7.36 (dd, 1H), 7.88 (d, 1H), 8.05-8.23 (m, 2H), 8.28-8.38 (m, 1H), 8.50-8.64 (m, 2H), 8.69 (s, 1H), 8.77 (d, 1H).

Compound No. 10: (250 MHz) 1.27-1.70 (m, 6H), 2.49-2.87 (m, 6H), 3.40-3.58 (m, 2H), 4.14 (d, 2H), 5.84 (s, 2H), 6.44 (d, 1H), 7.07 (t, 1H), 7.15 (t, 1H), 7.36 (dd, 1H), 7.40-7.50 (m, 2H), 7.77 (d, 2H), 7.88 (d, 1H), 8.03 (dd, 1H), 8.13 (dd, 1H), 8.38 (t, 1H), 8.63 (d, 1H), 8.81-9.04 (m, 2H), 11.15 (s, 1H).

Compound No. 11: (250 MHz) 1.20 (t, 3H), 1.29-1.66 (m, 6H), 2.11-2.47 (m, 6H), 3.25-3.40 (m, 2H), 3.42-3.62 (m, 2H), 4.11 (d, 2H), 5.76 (s, 2H), 6.43 (d, 1H), 6.61 (br. s., 1H), 6.97 (d, 1H), 7.12 (d, 1H), 7.35 (d, 1H), 7.62 (d, 1H), 7.77-8.05 (m, 3H), 8.28-8.48 (m, 2H), 8.91 (s, 1H).

Compound No. 12: (250 MHz) 1.22 (t, 3H), 1.57-1.86 (m, 4H), 2.43-2.52 (m, 4H), 2.57 (t, 2H), 3.29-3.43 (m, 2H), 3.43-3.60 (m, 2H), 4.13 (d, 2H), 5.83 (s, 2H), 6.44 (d, 1H), 6.99 (t, 1H), 7.15 (d, 1H), 7.35 (dd, 1H), 7.80-8.04 (m, 4H), 8.28 (t, 1H), 8.35-8.48 (m, 2H), 8.51 (br. s., 1H).

Compound No. 13: (250 MHz) 1.22 (t, 3H), 2.75 (d, 3H), 3.43-3.66 (m, 2H), 4.13 (d, 2H), 5.83 (s, 2H), 6.44 (d, 1H), 6.98 (t, 1H), 7.15 (d, 1H), 7.35 (dd, 1H), 7.82-8.04 (m, 4H), 8.28 (t, 1H), 8.35-8.62 (m, 3H).

Compound No. 14: (250 MHz) 1.22 (t, 3H), 3.27 (s, 3H), 3.35-3.62 (m, 6H), 4.13 (d, 2H), 5.83 (s, 2H), 6.44 (d, 1H), 6.97 (t, 1H), 7.15 (d, 1H), 7.37 (dd, 1H), 7.82-8.07 (m, 4H), 8.28 (t, 1H), 8.50 (br. s., 3H).

Compound No. 16: (400 MHz) 1.22 (t, 3H), 2.56 (t, 2H), 2.66-2.75 (m, 4H), 2.82-3.04 (m, 4H), 3.34-3.41 (m, 2H), 3.45-3.60 (m, 2H), 4.13 (d, 2H), 5.87 (s, 2H), 6.44 (d, 1H), 6.99 (t, 1 H), 7.15 (d, 1H), 7.35 (dd, 1H), 7.82-7.99 (m, 4H), 8.27 (t, 1H), 8.34-8.47 (m, 2H), 8.50 (d, 1H).

Compound No. 17: (250 MHz) 1.22 (t, 3H), 1.29-1.62 (m, 6H), 2.04 (s, 3H), 2.29-2.47 (m, 6H), 3.30-3.41 (m, 2H), 3.44-3.60 (m, 2H), 4.13 (d, 2H), 5.62 (s, 2H), 6.96 (t, 1H), 7.16 (d, 1 H), 7.22 (s, 1H), 7.76 (s, 1H), 7.82-8.06 (m, 3H), 8.28 (t, 1H), 8.33-8.46 (m, 2H), 8.48 (d, 1 H).

Compound No. 18: (250 MHz) 0.32-0.57 (m, 1H), 0.78 (d, 6H), 1.18 (t, 3H), 1.35-1.70 (m, 5H), 2.39 (t, 2H), 2.79 (d, 2H), 3.22-3.38 (m, 2H), 3.41-3.56 (m, 2H), 4.09 (d, 2H), 5.78 (s, 2 H), 6.39 (d, 1H), 6.92 (t, 1H), 7.11 (d, 1H), 7.30 (dd, 1H), 7.74-7.98 (m, 4H), 8.23 (t, 1H), 8.28-8.42 (m, 2H), 8.44 (d, 1H).

Compound No. 19: (250 MHz) 1.11 (d, 6H), 1.02-1.32 (m, 3H), 1.21 (t, 3H), 1.30-1.73 (m, 3H), 2.37-2.51 (m, 2H), 2.59-2.78 (m, 2H), 3.11-3.20 (m, 2H), 3.42-3.59 (m, 2H), 4.13 (d, 2 H), 5.83 (s, 2H), 6.44 (d, 1H), 6.97 (t, 1H), 7.16 (d, 1H), 7.35 (dd, 1H), 7.83-8.01 (m, 4H), 8.28 (t, 1H), 8.35-8.59 (m, 3H).

Compound No. 20: (250 MHz) 0.94-1.13 (m, 4H), 1.22 (t, 3H), 1.30-1.68 (m, 6H), 2.41-2.65 (m, 6H), 3.31-3.68 (m, 4H), 5.70 (s, 2H), 6.43 (d, 1H), 6.67 (br. s., 1H), 7.16 (d, 1H), 7.29 (d, 1 H), 7.73-8.07 (m, 4H), 8.19-8.34 (m, 1H), 8.42 (br. s., 2H), 8.54 (s, 1H).

Compound No. 21: (250 MHz) 1.13 (t, 3H), 1.27-1.69 (m, 6H), 2.16-2.47 (m, 6H), 3.32-3.43 (m, 4H), 4.11 (d, 2H), 5.81 (s, 2H), 6.43 (d, 1H), 6.61-8.82 (m, 2H), 7.23 (d, 2H), 7.35 (d, 1H), 7.87 (s, 1H), 7.92 (d, 1H), 8.27 (br. s., 1H), 8.41 (br. s., 1H), 9.03 (s, 1H).

Compound No. 22: (250 MHz) 1.19 (t, 3H), 1.30-1.64 (m, 6H), 2.31-2.50 (m, 6H), 3.32-3.42 (m, 2H), 3.41-3.58 (m, 2H), 4.13 (d, 2H), 5.84 (s, 2H), 6.44 (d, 1H), 6.89-7.06 (m, 2H), 7.35 (dd, 1H), 7.70-7.83 (m, 1H), 7.88 (d, 1H), 7.96 (d, 1H), 8.09 (t, 1H), 8.35 (t, 1H), 8.43 (t, 1H), 8.67 (d, 1H).

Compound No. 23: (250 MHz) 1.22 (t, 3H), 1.33-1.68 (m, 6H), 2.52-2.92 (m, 6H), 3.15-3.27 (m, 2H), 3.34-3.50 (m, 2H), 4.12 (d, 2H), 5.83 (s, 2H), 6.44 (d, 1H), 6.80-6.90 (m, 2H), 6.94 (t, 1H), 7.35 (dd, 1H), 7.48 (d, 1H), 7.52-7.66 (m, 2H), 7.78-7.93 (m, 2H), 8.13-8.37 (m, 2 H), 8.43 (d, 1H).

Compound No. 24: (250 MHz) 0.39-0.51 (m, 2H), 0.51-0.59 (m, 2H), 0.59-0.75 (m, 2H), 0.75-0.90 (m, 2H), 2.68-2.86 (m, 1H), 2.86-3.09 (m, 1H), 4.13 (d, 2H), 5.83 (s, 2H), 6.44 (d, 1H), 6.98 (t, 1H), 7.21 (d, 1H), 7.35 (dd, 1H), 7.83-8.13 (m, 4H), 8.29 (t, 1H), 8.38-8.65 (m, 3H).

Compound No. 25: (250 MHz) 0.35-0.55 (m, 2H), 0.69-0.84 (m, 2H), 1.41-2.01 (m, 8H), 2.85-3.05 (m, 1H), 4.13 (d, 2H), 4.13-4.26 (m, 1H), 5.83 (s, 2H), 6.44 (d, 1H), 6.98 (t, 1H), 7.22 (d, 1H), 7.35 (dd, 1H), 7.84-8.08 (m, 4H), 8.22-8.37 (m, 2H), 8.44-8.57 (m, 2H)

Compound No. 26: (250 MHz) 0.37-0.55 (m, 2H), 0.71-0.83 (m, 2H), 0.89 (t, 3H), 1.18-1.41 (m, 2H), 1.41-1.63 (m, 2H), 2.84-3.05 (m, 1H), 3.21 (q, 2H), 4.13 (d, 2H), 5.86 (s, 2H), 6.45 (d, 1H), 6.98 (t, 1H), 7.23 (d, 1H), 7.36 (dd, 1H), 7.83-8.09 (m, 4H), 8.29 (t, 1H), 8.39-8.68 (m, 3H)

Compound No. 27: (250 MHz) 1.22 (t, 3H), 1.29-1.58 (m, 6H), 2.28-2.47 (m, 6H), 3.30-3.41 (m, 2H), 3.43-3.62 (m, 2H), 4.37 (d, 2H), 7.16 (d, 1H), 7.23 (t, 1H), 7.39 (dd, 1H), 7.69-7.79 (m, 1H), 7.82-8.03 (m, 3H), 8.26 (t, 1H), 8.31-8.46 (m, 2H), 8.49 (dd, 1H), 8.56 (d, 1H), 8.64 (d, 1H).

Compound No. 28: (250 MHz) 1.22 (t, 3), 1.23-1.6 (m, 6), 2.30-2.50 (m, 6), 2.75 (d, 3), 3.34 (m, 2), 3.52 (qui, 2), 4.13 (d, 2), 6.39 (q, 1), 6.43 (d, 1), 6.98 (t, 1), 7.16 (d, 1), 7.37 (dd, 1), 7.80-8.02 (m, 4), 8.28 (t, 1), 8.37 (t, 1), 8.41 (t, 1), 8.49 (d, 1).

Compound No. 29: (250 MHz) 1.22 (t, 3H), 1.29-1.60 (m, 6H), 2, 31-247 (m, 6H), 3.00 (s, 6H), 3.26-3.42 (m, 2H), 3.45-3.60 (m, 2H), 4.18 (d, 2H), 6.64 (d, 1H), 7.01 (s, 1H), 7.16 (d, 1H), 7.49 (dd, 1H), 7.81-8.01 (m, 3H), 8.06 (d, 1H), 8.28 (t, 1H), 8.33-8.46 (m, 2H), 8.51 (d, 1H)

Compound No. 30: (250 MHz) 1.22 (t, 3H), 1.30-1.63 (m, 6H), 2.29-2.47 (m, 6H), 3.29-3.40 (m, 2H), 3.44-3.63 (m, 2H), 4.22 (d, 2H), 5.32 (s, 2H), 6.86 (s, 1H), 7.03-7.22 (m, 2H), 7.71 (s, 1H), 7.79-8.02 (m, 4H), 8.28 (t, 1H), 8.33-8.51 (m, 2H), 8.58 (s, 1H)

Compound No. 31: (500 MHz) 1.23 (t, 3H), 1.34-1.43 (m, 2H), 1.45-1.57 (m, 4H), 2.31-2.49 (m, 6H), 3.31-3.30 (m, 2H), 3.46-3.59 (m, 2H), 4.43 (d, 2H), 7.17 (d, 1H), 7.27 (t, 1H), 7.62-7.76 (m, 1H), 7.88 (dd, 1H), 7.91-8.02 (m, 2H), 8.24 (t, 1H), 8.38 (t, 1H), 8.42 (t, 1H), 8.46 (s, 1H), 8.49 (d, 1H), 8.69 (d, 1H)

Compound No. 32: (400 MHz) 1.22 (t, 3H), 1.33-1.61 (m, 6H), 2.31 (s, 3H), 2.51 (s, 6H), 3.34-3.43 (m, 2H), 3.46-3.60 (m, 2H), 4.34 (d, 2H), 716 (d, 1H), 7.21 (t, 1H), 7.54 (s, 1H), 7.87 (d, 1H), 7.90-8.05 (m, 2H), 8.21-8.29 (m, 1H), 8.29-8.37 (m, 2H), 8.37-8.49 (m, 2H), 8.62 (d, 1H)

Compound No. 33: (250 MHz) 1.22 (t, 3H), 1.31-1.59 (m, 6H), 2.22-2.47 (m, 6H), 3.29-3.41 (m, 2H), 3.53 (m, 2H), 5.67 (s, 2H), 6.45 (d, 1H), 7.17 (d, 1H), 7.51 (dd, 1H), 7.80-8.05 (m, 4H), 8.27 (t, 1H), 8.41 (d, 2H), 8.70 (br. s., 2H).

Compound No. 34: (400 MHz) 1.22 (t, 3H), 1.40 (d, 2H), 1.53 (quin, 4H), 2.41-2.60 (m, 6H), 2.45 (s, 3H), 3.34-3.42 (m, 2H), 3.47-3.57 (m, 2H), 4.32 (d, 2H), 7.11-7.21 (m, 2H), 7.23 (d, 1H), 7.61 (dd, 1H), 7.87 (d, 1H), 7.90-8.03 (m, 2H), 8.25 (t, 1H), 8.41 (s, 3H), 8.60 (d, 1H)

Compound No. 35: (400 MHz) 1.22 (t, 3H), 1.32-1.45 (m, 2H), 1.64-1.78 (m, 2H), 2.06 (t, 2H), 2.43 (t, 2H), 2.64-2.77 (m, 2H), 3.32 (s, 2H), 3.37-3.48 (m, 1H), 3.48-3.57 (m, 2H), 4.13 (d, 2H), 4.52 (d, 1H), 5.83 (s, 2H), 6.43 (d, 1H), 6.97 (t, 1H), 7.15 (d, 1H), 7.34 (dd, 1H), 7.86 (d, 2H), 7.89-7.99 (m, 2H), 8.27 (t, 1H), 8.33-8.45 (m, 2H), 8.49 (d, 1H)

Compound No. 36: (250 MHz) 0.94-1.15 (m, 1H), 1.22 (t, 3H), 1.30-1.52 (m, 1H), 1.52-1.69 (m, 1H), 1.80-1.97 (m, 3H), 2.44 (t, 2H), 2.62-2.77 (m, 1H), 2.85 (dd, 1H), 3.28-3.39 (m, 2H), 3.39-3.62 (m, 3H), 4.13 (d, 2H), 4.56 (d, 1H), 5.83 (s, 2H), 6.44 (d, 1H), 6.99 (t, 1H), 7.16 (d, 1H), 7.35 (dd, 1H), 7.82-8.03 (m, 4H), 8.28 (t, 1H), 8.33-8.46 (m, 2H), 8.51 (d, 1H)

Compound No. 37: (400 MHz) 0.99 (d, 12), 1.22 (t, 3), 2.51 (m, 2), 2.99 (m, 2), 3.19 (q, 2), 3.53 (qui, 2), 4.13 (d, 2), 5.83 (s, 2), 6.43 (d, 1), 6.97 (t, 1), 7.15 (d, 1), 7.34 (dd, 1), 7.80-8.00 (broad unresolved peak, 4); 8.27 (t, 1), 8.37 (t, 1), 8.42-8.54 (broad unresolved peak, 2)

Compound No. 38: (400 MHz) 1.22 (t, 3), 1.40 (m, 2), 1.82 (m, 2), 2.12 (t, 2), 2.44 (t, 2), 2.72 (m, 2), 3.15 (sep, 1), 3.22 (s, 3), 3.32 (m, 2), 3.52 (qui, 2), 4.15 (d, 2), 5.84 (s, 2), 6.43 (d, 1), 6.97 (t, 1), 7.15 (d, 1), 7.34 (dd, 1), 7.80-8.00 (m, 4), 8.27 (t, 1), 8.37 (t, 1), 8.40 (t, 1), 8.49 (d, 1).

Compound No. 39: (250 MHz) 1.22 (t, 3H), 1.30-1.57 (m, 6H), 1.66 (t, 2H), 2.18-2.41 (m, 6H), 3.16-3.29 (m, 2H), 3.40-3.60 (m, 2H), 4.13 (d, 2H), 5.83 (s, 2H), 6.44 (d, 1H), 6.97 (t, 1 H), 7.15 (d, 1H), 7.35 (dd, 1H), 7.81-8.03 (m, 4H), 8.28 (t, 1H), 8.36-8.58 (m, 3H).

Compound No. 40: (400 MHz) 1.22 (t, 3), 1.30-1.59 (m, 10), 2.23 (t, 2), 2.28 (br. s., 4), 3.23 (q, 2); 3.52 (qui, 2), 4.13 (d, 2), 5.83 (s, 2), 6.43 (d, 1), 6.99 (t, 1), 7.14 (d, 1), 7.34 (dd, 1), 7.82-8.00 (m, 4), 8.26 (t, 1), 8.43 (m, 2), 8.51 (br. s. 1).

Compound No. 41: (400 MHz) 1.22 (t, 3H), 2.14 (s, 3H), 2.18-2.49 (m, 10H), 3.31-3.39 (m, 2H), 3.44-3.58 (m, 2H), 4.13 (d, 2H), 5.83 (s, 2H), 6.43 (d, 1H), 6.97 (t, 1H), 7.15 (d, 1H), 7.34 (dd, 1H), 7.81-7.89 (m, 2H), 7.89-7.98 (m, 2H), 8.27 (t, 1H), 8.32-8.45 (m, 2H), 8.49 (d, 1H)

Compound No. 42: (250 MHz) 1.22 (t, 3H), 2.02 (br. s., 1H), 2.27-2.38 (m, 4H), 2.41 (t, 2H), 2.62-2.74 (m, 4H), 3.22-3.41 (m, 2H), 3.44-3.59 (m, 2H), 4.13 (d, 2H), 5.83 (s, 2H), 6.44 (d, 1H), 7.00 (t, 1H), 7.16 (d, 1H), 7.35 (dd, 1H), 7.82-8.02 (m, 4H), 8.28 (t, 1H), 8.33-8.46 (m, 2H), 8.51 (d, 1H)

Compound No. 43: ¹H NMR (500 MHz, d₆-DMSO) δ ppm 1.24 (t, 3H), 1.35-1.44 (m, 2H), 1.51 (quin, 4H), 2.33-2.50 (m, 6H), 3.33-3.41 (m, 2H), 3.50-3.59 (m, 2H), 6.48-6.56 (m, 3H), 6.86 (d, 1H), 7.22 (d, 1H), 7.49 (d, 1H), 7.66 (dd, 1H), 7.90-8.06 (m, 3H), 8.16 (d, 1H), 8.31 (t, 1H), 8.41 (q, 2H), 9.88 (s, 1H).

Compound No. 44: ¹H NMR (250 MHz, d₆-DMSO) δ ppm 1.23 (t, 3H), 1.31-1.64 (m, 6H), 2.23-2.47 (m, 6H), 3.29-3.41 (m, 2H), 3.46-3.65 (m, 2H), 7.21 (s, 1H), 7.26 (d, 1H), 7.51 (dd, 1H), 7.69 (d, 1H), 7.93-8.13 (m, 4H), 8.31 (t, 1H), 8.37-8.47 (m, 2H), 8.62 (d, 1H), 885 (d, 1H), 10.17 (s, 1H).

Compound No. 45: ¹H NMR (250 MHz, d₆-DMSO) δ ppm 1.22 (t, 7H), 1.96-2.26 (m, 2H), 3.16 (d, 4H), 3.40 (t, 2H), 3.52 (ddt, 2H), 3.71 (d, 2H), 4.13 (d, 2H), 5.83 (s, 2H), 6.44 (d, 1H), 7.07 (t, 1H), 7.13 (d, 1H), 7.35 (dd, 1H), 7.73 (d, 1H), 7.80-7.96 (m, 3H), 8.28 (t, 1H), 8.55 (br. s., 2 H), 11.05 (br. s., 1H)

TABLE I

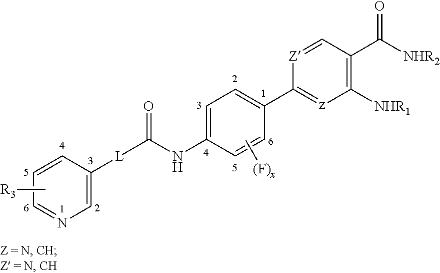

| Com- | | | | | Po- | | | | LC | M.p. | |
|---|---|---|---|---|---|---|---|---|---|---|---|
| pound | | | | | sition | | | MS | (meth- | (° C.)/ | |
| No. | R₃ | L* | Z/Z′ | x | of F | R₁ | R₂ | (MH⁺) | od) | NMR | Scheme |
| 1 | 6-NH₂ | CH₂NH | N/CH | 1 | 3-F | Et | 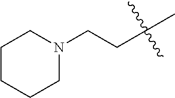 | 535 | 5.12 (TFA15) 0.79 (TFA3) | M.p. = 175- 177° C. NMR | 1 or 2 (for Ex. 1, Scheme 2 used) |
| 2 | 6-NH₂ | CH₂NH | N/CH | 2 | 2-F, 5-F | Et | 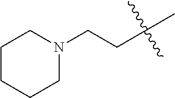 | 553 | 0.82 (TFA3) | NMR | 2 |
| 3 | 6-NH₂ | CH₂NH | N/N | 1 | 3-F | Et | 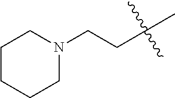 | 536 | 0.67 (TFA3) | NMR | 2 |
| 4 | 6-NH₂ | CH₂NH | N/CH | 1 | 3-F | 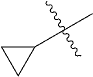 | 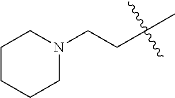 | 547 | 0.73 (TFA3) | NMR | 2 |
| 5 | 6-NH₂ | CH₂NH | N/CH | 1 | 3-F | 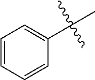 | 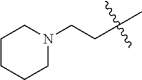 | 583 | 0.96 (TFA3) | NMR | 2 |
| 6 | 6-NH₂ | CH₂NH | N/CH | 1 | 3-F | Et | —CH₂CH₂NH(i-Pr) | 509 | 0.76 | NMR | 1 |
| | | | | | | | | | (TFA3) | | |
| 7 | 6-NH₂ | CH₂NH | N/CH | 1 | 3-F | Et | 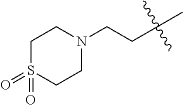 | 585 | 0.76 (TFA3) | NMR | 2 |
| 8 | 6-NH₂ | CH₂NH | N/CH | 1 | 3-F | Et | —CH₂CH₂OH | 468 | 0.72 | NMR | 1; see |
| | | | | | | | | | (TFA3) | | Ex. 2 |
| 9 | 6-NH₂ | CH₂NH | N/N | 1 | 3-F | 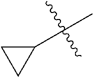 | 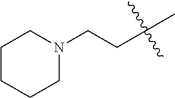 | 548 | 0.71 (TFA3) | NMR | 3 |
| 10 | 6-NH₂ | CH₂NH | N/N | 1 | 3-F | 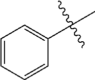 | 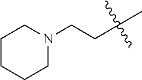 | 584 | 0.88 (TFA3) | NMR | 3 |
| 11 | 6-NH₂ | CH₂NH | N/CH | 1 | 2-F | Et | 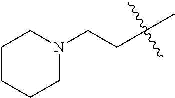 | 535 | 0.7 (TFA) | NMR | 2 |
| 12 | 6-NH₂ | CH₂NH | N/CH | 1 | 3-F | Et | 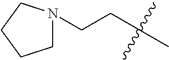 | 521 | 0.74 (TFA3) | NMR | 3 |
| 13 | 6-NH₂ | CH₂NH | N/CH | 1 | 3-F | Et | —Me | 438 | 0.82 | NMR | 3 |
| | | | | | | | | | (TFA3) | | |
| 14 | 6-NH₂ | CH₂NH | N/CH | 1 | 3-F | Et | —CH₂CH₂OMe | 482 | 0.89 | NMR | 3 |
| | | | | | | | | | (TFA3) | | |
| 15 | 6-NH₂ | CH₂NH | N/CH | 1 | 3-F | Et | 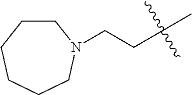 | 549 | 0.85 (TFA3) | NMR | 3; see Ex. 3 |
| 16 | 6-NH₂ | CH₂NH | N/CH | 1 | 3-F | Et | 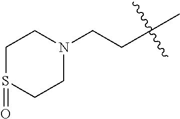 | 569 | 0.67 (TFA3) | NMR | 3 |
| 17 | 5-Me, 6-NH₂ | CH₂NH | N/CH | 1 | 3-F | Et | 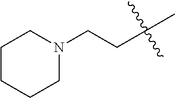 | 549 | 0.82 (TFA3) | NMR | 2 |
| 18 | 6-NH₂ | CH₂NH | N/CH | 1 | 3-F | Et | 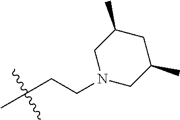 | 563 | 0.93 (TFA3) | NMR | 3 |
| 19 | 6-NH₂ | CH₂NH | N/CH | 1 | 3-F | Et | 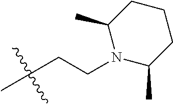 | 563 | 0.87 (TFA3) | NMR | 3 |
| 20 | 6-NH₂ | CH₂CH₂NH | N/CH | 1 | 3-F | Et | 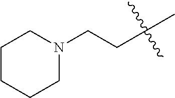 | 549 | 0.81 (TFA3) | NMR | 2 |
| 21 | 6-NH₂ | CH₂NH | N/CH | 2 | 2-F, 6-F | Et | 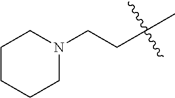 | 553 | 0.71 (TFA3) | NMR | 2 |
| 22 | 6-NH₂ | CH₂NH | N/CH | 2 | 2-F, 3-F | Et | 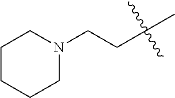 | 553 | 0.78 (TFA3) | NMR | 2 |
| 23 | 6-NH₂ | CH₂NH | CH/CH | 1 | 3-F | Et | 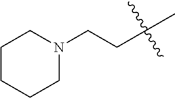 | 534 | 0.79 (TFA3) | NMR | 2 |
| 24 | 6-NH₂ | CH₂NH | N/CH | 1 | 3-F | 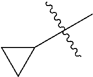 | 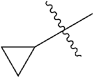 | 476 | 0.86 (TFA3) | NMR | 3 |
| 25 | 6-NH₂ | CH₂NH | N/CH | 1 | 3-F | 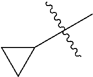 | 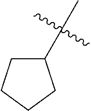 | 504 | 1.10 (TFA3) | NMR | 3 |
| 26 | 6-NH₂ | CH₂NH | N/CH | 1 | 3-F | 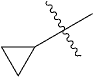 | -(n-Bu) | 492 | 1.09 (TFA3) | NMR | 3 |
| 27 | H | CH₂NH | N/CH | 1 | 3-F | Et | 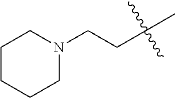 | 520 | 0.78 (TFA3) | NMR | 2 |
| 28 | 6- NHMe | CH₂NH | N/CH | 1 | 3-F | Et | 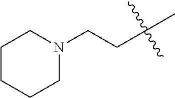 | 549 | 0.81 (TFA3) | NMR | 2 |
| 29 | 6- NMe₂ | CH₂NH | N/CH | 1 | 3-F | Et | 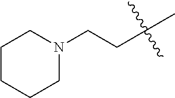 | 563 | 0.83 (TFA3) | NMR | 2 |
| 30 | 5-NH₂ | CH₂NH | N/CH | 1 | 3-F | Et | 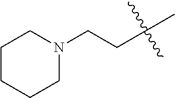 | 535 | 0.77 (TFA3) | NMR | 2 |
| 31 | 5-F | CH₂NH | N/CH | 1 | 3-F | Et | 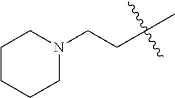 | 538 | 0.97 (TFA3) | NMR | 2 |
| 32 | 5-Me | CH₂NH | N/CH | 1 | 3-F | Et | 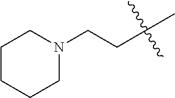 | 534 | 0.81 (TFA3) | NMR | 2 |
| 33 | 6-NH₂ | NH | N/CH | 1 | 3-F | Et | 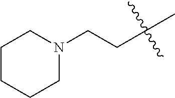 | 521 | 0.81 (TFA3) | NMR | 2 |
| 34 | 6-Me | CH₂NH | N/CH | 1 | 3-F | Et | 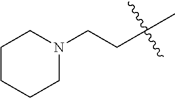 | 534 | 0.79 (TFA3) | NMR | 2 |
| 35 | 6-NH₂ | CH₂NH | N/CH | 1 | 3-F | Et | 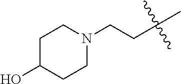 | 551 | 1.1 (TFA6) | NMR | 3 |
| 36 | 6-NH₂ | CH₂NH | N/CH | 1 | 3-F | Et | 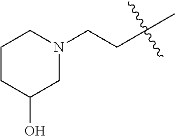 | 551 | 0.72 (TFA3) | NMR | 3 |
| 37 | 6-NH₂ | CH₂NH | N/CH | 1 | 3-F | Et | —CH₂CH₂N(i-Pr)₂ | 551 | 0.87 | NMR | 3 |
| | | | | | | | | | (TFA3) | | |
| 38 | 6-NH₂ | CH₂NH | N/CH | 1 | 3-F | Et | 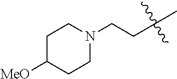 | 565 | 0.79 (TFA3) | NMR | 3 |
| 39 | 6-NH₂ | CH₂NH | N/CH | 1 | 3-F | Et | 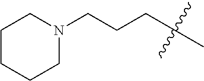 | 549 | 0.79 (TFA3) | NMR | 3 |
| 40 | 6-NH₂ | CH₂NH | N/CH | 1 | 3-F | Et | 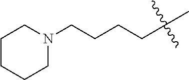 | 563 | 0.79 (TFA3) | NMR | 3 |
| 41 | 6-NH₂ | CH₂NH | N/CH | 1 | 3-F | Et | 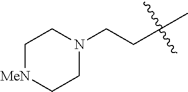 | 550 | 0.67 (TFA3) | NMR | 3 |
| 42 | 6-NH₂ | CH₂NH | N/CH | 1 | 3-F | Et | 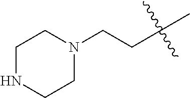 | 536 | 0.65 (TFA3) | NMR | 3 |
| 43 | 6-NH₂ | —CH═CH— | N/CH | 1 | 3-F | Et | 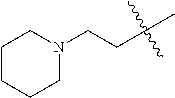 | 532 | 1.17 (TFA3) | NMR | |
| 44 | H | —CH═CH— | N/CH | 1 | 3-F | Et | 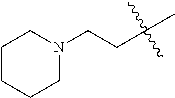 | 517 | 0.89 (TFA3) | NMR | |
| 45 | 6-NH₂ | CH₂NH | N/CH | 1 | 3-F | Et | 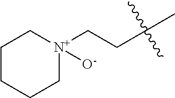 | 551 | 0.82 (TFA3) | NMR | |

*for L, NH bonded to the C═O;
**n-Bu: n-butyl; t-Bu: tert-butyl; i-Pr: isopropyl
The compounds in Table I have the chemical names (obtained from the Autonom ® software):
6-{4-[3-(6-Aminopyridin-3-ylmethyl)ureido]-3-fluorophenyl}-2-ethylamino-N-[2-(piperidin-1-yl)ethyl]nicotinamide (compound No. 1)
6-{4-[3-(6-Aminopyridin-3-ylmethyl)ureido]-2,5-difluorophenyl}-2-ethylamino-N-[2-(piperidin-1-yl)ethyl]nicotinamide (No. 2)
2-{4-[3-(6-Aminopyridin-3-ylmethyl)ureido]-3-fluorophenyl}-4-(ethylamino)pyrimidine-5-carboxylic acid [2-(piperidin-1-yl)ethyl]amide (No. 3)
6-{4-[3-(6-Aminopyridin-3-ylmethyl)ureido]-3-fluorophenyl}-2-cyclopropylamino-N-[2-(piperidin-1-yl)ethyl]nicotinamide (No. 4)
6-{4-[3-(6-Aminopyridin-3-ylmethyl)ureido]-3-fluorophenyl}-2-phenylamino-N-[2-(piperidin-1-yl)ethyl]nicotinamide (No. 5)
6-{4-[3-(6-Aminopyridin-3-ylmethyl)ureido]-3-fluorophenyl}-2-ethylamino-N-[2-(isopropylamino)ethyl]nicotinamide (No. 6)
6-{4-[3-(6-Aminopyridin-3-ylmethyl)ureido]-3-fluorophenyl}-N-[2-(1,1-dioxothiomorpholin-4-yl)ethyl]-2-(ethylamino)nicotinamide (No. 7)
6-{4-[3-(6-Aminopyridin-3-ylmethyl)ureido]-3-fluorophenyl}-2-ethylamino-N-(2-hydroxy-ethyl)nicotinamide (No. 8)
2-{4-[3-(6-Aminopyridin-3-ylmethyl)ureido]-3-fluorophenyl}-4-(cyclopropylamino)pyrimidine-5-carboxylic acid [2-(piperidin-1-yl)ethyl]amide (No. 9)
2-{4-[3-(6-Aminopyridin-3-ylmethyl)ureido]-3-fluorophenyl}-4-(phenylamino)pyrimidine-5-carboxylic acid [2-(piperidin-1-yl)ethyl]amide (No. 10)
6-{4-[3-(6-Aminopyridin-3-ylmethyl)ureido]-2-fluorophenyl}-2-ethylamino-N-[2-(piperidin-1-yl)ethyl]nicotinamide (No. 11)
6-{4-[3-(6-Aminopyridin-3-ylmethyl)ureido]-3-fluorophenyl}-2-ethylamino-N-[2-(pyrrolidin-1-yl)ethyl]nicotinamide (No. 12)
6-{4-[3-(6-Aminopyridin-3-ylmethyl)ureido]-3-fluorophenyl}-2-ethylamino-N-methyl-nicotinamide (No. 13)
6-{4-[3-(6-Aminopyridin-3-ylmethyl)ureido]-3-fluorophenyl}-2-ethylamino-N-(2-methoxy-ethyl)nicotinamide (No. 14)
6-{4-[3-(6-Aminopyridin-3-ylmethyl)ureido]-3-fluorophenyl}-N-[2-(azepan-1-yl)ethyl]-2-(ethylamino)nicotinamide (No. 15)
6-{4-[3-(6-Aminopyridin-3-ylmethyl)ureido]-3-fluorophenyl}-2-ethylamino-N-[2-(1-oxo-thiomorpholin-4-yl)ethyl]nicotinamide (No. 16)
6-{4-[3-(6-Amino-5-methylpyridin-3-ylmethyl)ureido]-3-fluorophenyl}-2-ethylamino-N-[2-(piperidin-1-yl)ethyl]nicotinamide (No. 17)
6-{4-[3-(6-Aminopyridin-3-ylmethyl)ureido]-3-fluorophenyl)-N-[2-(cis-3,5-dimethylpiperidin-1-yl)ethyl]-2-(ethylamino)nicotinamide (No. 18)
6-{4-[3-(6-Aminopyridin-3-ylmethyl)ureido]-3-fluorophenyl]-N-[2-(cis-2,6-dimethylpiperidin-1-yl)ethyl]-2-(ethylamino)nicotinamide (No. 19)
6-(4-{3-[2-(6-Aminopyridin-3-yl)ethyl]ureido}-3-fluorophenyl)-2-ethylamino-N-[2-(piperidin-1-yl)ethyl]nicotinamide (No. 20)
6-{4-[3-(6-Aminopyridin-3-ylmethyl)ureido]-2,6-difluorophenyl}-2-ethylamino-N-[2-(piperidin-1-yl)ethyl]nicotinamide (No. 21)
6-{4-[3-(6-Aminopyridin-3-ylmethyl)ureido]-2,3-difluorophenyl}-2-ethylamino-N-[2-(piperidin-1-yl)ethyl]nicotinamide (No. 22)
4′-[3-(6-Aminopyridin-3-ylmethyl)ureido]-3-ethylamino-3′-fluorobiphenyl-4-carboxylic acid [2-(piperidin-1-yl)ethyl]amide (No. 23)
6-{4-[3-(6-Aminopyridin-3-ylmethyl)ureido]-3-fluorophenyl}-N-cyclopropyl-2-(cyclopropylamino)nicotinamide (No. 24)
6-{4-[3-(6-Aminopyridin-3-ylmethyl)ureido]-3-fluorophenyl}-N-cyclopentyl-2-(cyclopropylamino)nicotinamide (No. 25)
6-{4-[3-(6-Aminopyridin-3-ylmethyl)ureido]-3-fluorophenyl}-N-butyl-2-(cyclopropylamino)nicotinamide (No. 26)
2-Ethylamino-6-{3-fluoro-4-[3-(pyridin-3-ylmethyl)ureido]phenyl}-N-[2-(piperidin-1-yl)ethyl]nicotinamide (No. 27)
2-Ethylamino-6-{3-fluoro-4-[3-(6-(methylamino)pyridin-3-ylmethyl)ureido]phenyl}-N-[2-(piperidin-1-yl)ethyl]nicotinamide (No. 28)
6-{4-[3-(6-(Dimethylamino)pyridin-3-ylmethyl)ureido]-3-fluorophenyl}-2-ethylamino-N-[2-(piperidin-1-yl)ethyl]nicotinamide (No. 29)
6-{4-[3-(5-Aminopyridin-3-ylmethyl)ureido]-3-fluorophenyl}-2-ethylamino-N-[2-(piperidin-1-yl)ethyl]nicotinamide (No. 30)
2-Ethylamino-6-{3-fluoro-4-[3-(5-fluoropyridin-3-ylmethyl)ureido]phenyl}-N-[2-(piperidin-1-yl)ethyl]nicotinamide (No. 31)
2-Ethylamino-6-{3-fluoro-4-[3-(5-methylpyridin-3-ylmethyl)ureido]phenyl}-N-[2-(piperidin-1-yl)ethyl]nicotinamide (No. 32)
6-{4-[3-(6-Aminopyridin-3-yl)ureido]-3-fluorophenyl}-2-ethylamino-N-[2-(piperidin-1-yl)ethyl]nicotinamide (No. 33)
2-Ethylamino-6-{3-fluoro-4-[3-(6-methylpyridin-3-ylmethyl)ureido]phenyl}-N-[2-(piperidin-1-yl)ethyl]nicotinamide (No. 34)
6-{4-[3-(6-Aminopyridin-3-ylmethyl)ureido]-3-fluorophenyl}-2-ethylamino-N-[2-(4-hydroxy-piperidin-1-yl)ethyl]nicotinamide (No. 35)
6-{4-[3-(6-Aminopyridin-3-ylmethyl)ureido]-3-fluorophenyl}-2-ethylamino-N-[2-(3-hydroxy-piperidin-1-yl)ethyl]nicotinamide (No. 36)
6-{4-[3-(6-Aminopyridin-3-ylmethyl)ureido]-3-fluorophenyl}-N-[2-(diisopropylamino)ethyl]-2-(ethylamino)nicotinamide (No. 37)
6-{4-[3-(6-Aminopyridin-3-ylmethyl)ureido]-3-fluorophenyl}-2-ethylamino-N-[2-(4-methoxy-piperidin-1-yl)ethyl]nicotinamide (No. 38)
6-{4-[3-(6-Aminopyridin-3-ylmethyl)ureido]-3-fluorophenyl}-2-ethylamino-N-[3-(piperidin-1-yl)propyl]nicotinamide (No. 39)
6-{4-[3-(6-Aminopyridin-3-ylmethyl)ureido]-3-fluorophenyl}-2-ethylamino-N-[4-(piperidin-1-yl)butyl]nicotinamide (No. 40)
6-{4-[3-(6-Aminopyridin-3-ylmethyl)ureido]-3-fluorophenyl}-2-ethylamino-N-[2-(4-methylpiperazin-1-yl)ethyl]nicotinamide (No. 41)
6-{4-[3-(6-Aminopyridin-3-ylmethyl)ureido]-3-fluorophenyl}-2-ethylamino-N-[2-(piperazin-1-yl)ethyl]nicotinamide (No. 42)
6-{4-[(E)-3-(6-Aminopyridin-3-yl)acryloylamino]-3-fluorophenyl}-2-ethylamino-N-[2-(piperidin-1-yl)ethyl]nicotinamide (No. 43)
2-Ethylamino-6-{3-fluoro-4-[(E)-3-(pyridin-3-yl)acryloylamino]phenyl}-N-[2-(piperidin-1-yl)ethyl]nicotinamide (No. 44)
6-{4-[3-(6-Aminopyridin-3-ylmethyl)ureido]-3-fluorophenyl}-2-ethylamino-N-[2-(1-oxy-piperidin-1-yl)ethyl]nicotinamide (No. 45)

The compounds described in Table I have formed the subject of pharmacological trials which make it possible to determine the anticancer activity. They were tested in vitro on the HCT116 tumour line (ATCC-CCL247). The cell proliferation and viability were determined in a test using 3-(4,5-dimethylthiazol-2-yl)-5-(3-carboxymethoxyphenyl)-2-(4-sulphophenyl)-2H-tetrazolium (MTS) according to Fujishita T. et al., Oncology, 2003, 64(4), 399-406. In this test, the mitochondrial ability of the living cells to convert MTS to a coloured compound is measured after incubating the test compound for 72 hours. The concentration of compound which results in a 50% loss of cell proliferation and viability is denoted IC₅₀.

For the compounds of Table I, an IC₅₀<1000 nM (1 μM) is found with regard to the HCT116 line. Some compounds (for example Nos. 1, 5, 11, 19, 27) even exhibit an activity <1 nM.

1. A compound of formula (I): 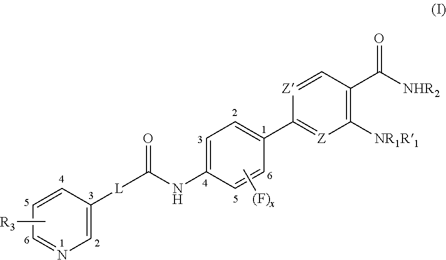
in which:
Z and Z′ represent N or CH;
x is an integer having the value 1 or 2, representing the number of fluorine atom(s) attached to the central phenyl nucleus;
L represents a —CH═CH— or —(CH₂)ₙNH— group in which the NH group is attached to the C═O and n is an integer having the value 0, 1 or 2;
R₁ represents a hydrogen atom or a (C₁-C₆)alkyl, (C₃-C₆)cycloalkyl or phenyl group;
R′₁ represents a hydrogen atom or a (C₁-C₆)alkyl group;
R₂ represents:
a (C₃-C₆)cycloalkyl group;
a (C₁-C₆)alkyl group, optionally substituted by:
one or more hydroxyl or (C₁-C₄)alkoxy groups;
an NRₐR_{b} group in which Rₐ and R_{b} represent, independently of one another, a hydrogen atom or a (C₁-C₆)alkyl group or form, together with the nitrogen atom to which they are connected, a (C₄-C₆)heterocycloalkyl group optionally comprising, in the ring, the —S(O)_{q}— group with q=0, 1 or 2 or the —NH— or —N(C₁-C₄ alkyl)- group and being optionally substituted by one or more substituent(s), which are identical to or different from one another when there are several of them, chosen from an —OH, (C₁-C₄)alkoxy or (C₁-C₄)alkyl group;
R₃ represents at least one substituent of the pyridine nucleus chosen from a hydrogen atom, a fluorine atom, a (C₁-C₄)alkyl group or an —NR_{c}R_{d} group in which R_{c} and R_{d} represent a hydrogen atom or a (C₁-C₄)alkyl group.

2. The compound according to claim 1, in which R₁ represents the cyclopropyl group or the phenyl group or a (C₁-C₆)alkyl group and R′₁ represents a hydrogen atom.

3. The compound according to claim 1, in which R′₁ represents a hydrogen atom.

4. The compound according to claim 1, in which R₂ represents:
the cyclopropyl or cyclopentyl group;
a (C₁-C₆)alkyl group, optionally substituted by:
one or more —OH or (C₁-C₄)alkoxy group(s);
the pyrrolidinyl 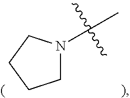
piperidinyl 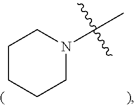
piperazinyl 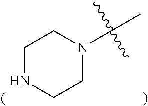
or N—(C₁-C₄ alkyl)piperazinyl 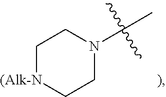
azepanyl 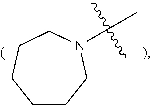
thiomorpholinyl 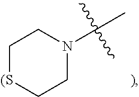
1-oxothiomorpholinyl 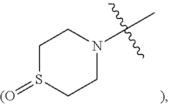
1,1-dioxothiomorpholinyl 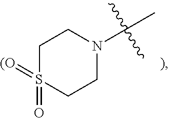
3-hydroxypiperidinyl 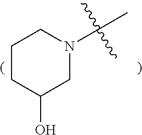
or 4-hydroxypiperidinyl 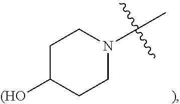
4-methoxypiperidinyl 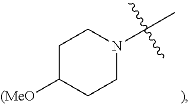
cis-3,5-dimethylpiperidinyl 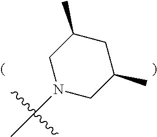
or cis-2,6-dimethylpiperidinyl 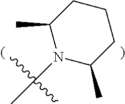
group.

5. The compound according to claim 1, in which R₃ is in the 5 and/or 6 position on the pyridine nucleus.

6. The compound according to claim 1, in which the number of R₃ substituents is equal to 1 and/or R₃ is in the 5 or 6 position on the pyridine nucleus.

7. The compound according to claim 1, in which R₃ is —NH₂.

8. The compound according to claim 1, in which n is equal to 1 or L represents the —CH═CH— group in the E or Z form.

9. The compound according to claim 1, in which Z and Z′ respectively represent N and CH, CH and CH or N and N.

10. The compound according to claim 1, in which x has the value 1.

11. The compound according to claim 1 of formula (I″): 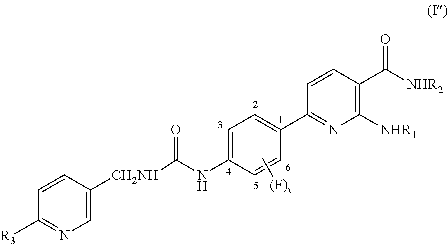
in which R₁ represents a (C₁-C₄)alkyl group, R₂ represents a (C₁-C₆)alkyl group optionally substituted by an —NRₐR_{b} group in which Rₐ and R_{b} form, together with the nitrogen atom to which they are connected, the (C₄-C₆)heterocycloalkyl group optionally comprising, in the ring, the —S(O)_{q}— group with q=0, 1 or 2 or the —NH— or —N(C₁-C₄ alkyl)- group, and x is an integer having the value 1 or 2, representing the number of fluorine atom(s) attached to the central phenyl nucleus.

12. The compound according to claim 11, in which the (C₄-C₆)heterocycloalkyl group is chosen from the pyrrolidinyl, piperidinyl, piperazinyl, N—(C₁-C₄ alkyl)piperazinyl, azepanyl, thiomorpholinyl, 1-oxothiomorpholinyl, 1,1-dioxothiomorpholinyl, 3-hydroxypiperidinyl or 4-hydroxypiperidinyl, 4-methoxypiperidinyl, cis-3,5-dimethylpiperidinyl or cis-2,6-dimethylpiperidinyl group.

13. The compound according to claim 1, in which x has the value 1 and the fluorine atom is in the 3 position.

14. The compound according to claim 1, in the form of the base or of an addition salt with an acid or in the form of a hydrate or of a solvate.

15. The compound according to claim 1, chosen from one of the following:
6-{4-[3-(6-Aminopyridin-3-ylmethyl)ureido]-3-fluorophenyl}-2-ethylamino-N-[2-(piperidin-1-yl)ethyl]nicotinamide
6-{4-[3-(6-Aminopyridin-3-ylmethyl)ureido]-2,5-difluorophenyl}-2-ethylamino-N-[2-(piperidin-1-yl)ethyl]nicotinamide
2-{4-[3-(6-Aminopyridin-3-ylmethyl)ureido]-3-fluorophenyl}-4-(ethylamino)pyrimidine-5-carboxylic acid[2-(piperidin-1-yl)ethyl]amide
6-{-4-[3-(6-Aminopyridin-3-ylmethyl)ureido]-3-fluorophenyl}-2-cyclopropylamino-N-[2-(piperidin-1-yl)ethyl]nicotinamide
6-{4-[3-(6-Aminopyridin-3-ylmethyl)ureido]-3-fluorophenyl}-2-phenylamino-N-[2-(piperidin-1-yl)ethyl]nicotinamide
6-{4-[3-(6-Aminopyridin-3-ylmethyl)ureido]-3-fluorophenyl}-2-ethylamino-N-[2-(isopropylamino)ethyl]nicotinamide
6-{4-[3-(6-Aminopyridin-3-ylmethyl)ureido]-3-fluorophenyl}-N-[2-(1,1-dioxothiomorpholin-4-yl)ethyl]-2-(ethylamino)nicotinamide
6-{4-[3-(6-Aminopyridin-3-ylmethyl)ureido]-3-fluorophenyl}-2-ethylamino-N-(2-hydroxy-ethyl)nicotinamide
2-{4-[3-(6-Aminopyridin-3-ylmethyl)ureido]-3-fluorophenyl}-4-(cyclopropylamino)pyrimidine-5-carboxylic acid[2-(piperidin-1-yl)ethyl]amide
2-{4-[3-(6-Aminopyridin-3-ylmethyl)ureido]-3-fluorophenyl}-4-(phenylamino)pyrimidine-5-carboxylic acid[2-(piperidin-1-yl)ethyl]amide
6-{4-[3-(6-Aminopyridin-3-ylmethyl)ureido]-2-fluorophenyl}-2-ethylamino-N-[2-(piperidin-1-yl)ethyl]nicotinamide
6-{4-[3-(6-Aminopyridin-3-ylmethyl)ureido]-3-fluorophenyl}-2-ethylamino-N-[2-(pyrrolidin-1-yl)ethyl]nicotinamide
6-{4-[3-(6-Aminopyridin-3-ylmethyl)ureido]-3-fluorophenyl}-2-ethylamino-N-methyl-nicotinamide
6-{4-[3-(6-Aminopyridin-3-ylmethyl)ureido]-3-fluorophenyl}-2-ethylamino-N-(2-methoxy-ethyl)nicotinamide
6-{4-[3-(6-Aminopyridin-3-ylmethyl)ureido]-3-fluorophenyl}-N-[2-(azepan-1-yl)ethyl]-2-(ethylamino)nicotinamide
6-{4-[3-(6-Aminopyridin-3-ylmethyl)ureido]-3-fluorophenyl}-2-ethylamino-N-[2-(1-oxo-thiomorpholin-4-yl)ethyl]nicotinamide
6-{4-[3-(6-Amino-5-methylpyridin-3-ylmethyl)ureido]-3-fluorophenyl}-2-ethylamino-N-[2-(piperidin-1-yl)ethyl]nicotinamide
6-{4-[3-(6-Aminopyridin-3-ylmethyl)ureido]-3-fluorophenyl}-N-[2-(cis-3,5-dimethylpiperidin-1-yl)ethyl]-2-(ethylamino)nicotinamide
6-{4-[3-(6-Aminopyridin-3-ylmethyl)ureido]-3-fluorophenyl}-N-[2-(cis-2,6-dimethylpiperidin-1-yl)ethyl]-2-(ethylamino)nicotinamide
6-(4-{3-[2-(6-Aminopyridin-3-yl)ethyl]ureido}-3-fluorophenyl)-2-ethylamino-N-[2-(piperidin-1-yl)ethyl]nicotinamide
6-{4-[3-(6-Aminopyridin-3-ylmethyl)ureido]-2,6-difluorophenyl}-2-ethylamino-N-[2-(piperidin-1-yl)ethyl]nicotinamide
6-{4-[3-(6-Aminopyridin-3-ylmethyl)ureido]-2,3-difluorophenyl}-2-ethylamino-N-[2-(piperidin-1-yl)ethyl]nicotinamide
4′-[3-(6-Aminopyridin-3-ylmethyl)ureido]-3-ethylamino-3′-fluorobiphenyl-4-carboxylic acid[2-(piperidin-1-yl)ethyl]amide
6-{4-[3-(6-Aminopyridin-3-ylmethyl)ureido]-3-fluorophenyl}-N-cyclopropyl-2-(cyclopropylamino)nicotinamide
6-{4-[3-(6-Aminopyridin-3-ylmethyl)ureido]-3-fluorophenyl}-N-cyclopentyl-2-(cyclopropylamino)nicotinamide
6-{4-[3-(6-Aminopyridin-3-ylmethyl)ureido]-3-fluorophenyl}-N-butyl-2-(cyclopropylamino)nicotinamide
2-Ethylamino-6-{3-fluoro-4-[3-(pyridin-3-ylmethyl)ureido]phenyl}-N-[2-(piperidin-1-yl)ethyl]nicotinamide
2-Ethylamino-6-{3-fluoro-4-[3-(6-(methylamino)pyridin-3-ylmethyl)ureido]phenyl}-N-[2-(piperidin-1-yl)ethyl]nicotinamide
6-{4-[3-(6-(Dimethylamino)pyridin-3-ylmethyl)ureido]-3-fluorophenyl}-2-ethylamino-N-[2-(piperidin-1-yl)ethyl]nicotinamide
6-{4-[3-(5-Aminopyridin-3-ylmethyl)ureido]-3-fluorophenyl}-2-ethylamino-N-[2-(piperidin-1-yl)ethyl]nicotinamide
2-Ethylamino-6-{3-fluoro-4-[3-(5-fluoropyridin-3-ylmethyl)ureido]phenyl}-N-[2-(piperidin-1-yl)ethyl]nicotinamide
2-Ethylamino-6-{3-fluoro-4-[3-(5-methylpyridin-3-ylmethyl)ureido]phenyl}-N-[2-(piperidin-1-yl)ethyl]nicotinamide
6-{4-[3-(6-Aminopyridin-3-yl)ureido]-3-fluorophenyl}-2-ethylamino-N-[2-(piperidin-1-yl)ethyl]nicotinamide
2-Ethylamino-6-{3-fluoro-4-[3-(6-methylpyridin-3-ylmethyl)ureido]phenyl}-N-[2-(piperidin-1-yl)ethyl]nicotinamide
6-{4-[3-(6-Aminopyridin-3-ylmethyl)ureido]-3-fluorophenyl}-2-ethylamino-N-[2-(4-hydroxy-piperidin-1-yl)ethyl]nicotinamide
6-{4-[3-(6-Aminopyridin-3-ylmethyl)ureido]-3-fluorophenyl}-2-ethylamino-N-[2-(3-hydroxy-piperidin-1-yl)ethyl]nicotinamide
6-{4-[3-(6-Aminopyridin-3-ylmethyl)ureido]-3-fluorophenyl}-N-[2-(diisopropylamino)ethyl]-2-(ethylamino)nicotinamide
6-{4-[3-(6-Aminopyridin-3-ylmethyl)ureido]-3-fluorophenyl}-2-ethylamino-N-[2-(4-methoxy-piperidin-1-yl)ethyl]nicotinamide
6-{4-[3-(6-Aminopyridin-3-ylmethyl)ureido]-3-fluorophenyl}-2-ethylamino-N-[3-(piperidin-1-yl)propyl]nicotinamide
6-{4-[3-(6-Aminopyridin-3-ylmethyl)ureido]-3-fluorophenyl}-2-ethylamino-N-[4-(piperidin-1-yl)butyl]nicotinamide
6-{4-[3-(6-Aminopyridin-3-ylmethyl)ureido]-3-fluorophenyl}-2-ethylamino-N-[2-(4-methylpiperazin-1-yl)ethyl]nicotinamide
6-{4-[3-(6-Aminopyridin-3-ylmethyl)ureido]-3-fluorophenyl}-2-ethylamino-N-[2-(piperazin-1-yl)ethyl]nicotinamide
6-{4-[(E)-3-(6-Aminopyridin-3-yl)acryloylamino]-3-fluorophenyl}-2-ethylamino-N-[2-(piperidin-1-yl)ethyl]nicotinamide
2-Ethylamino-6-{3-fluoro-4-[(E)-3-(pyridin-3-yl)acryloylamino]phenyl}-N-[2-(piperidin-1-yl)ethyl]nicotinamide
6-{4-[3-(6-Aminopyridin-3-ylmethyl)ureido]-3-fluorophenyl}-2-ethylamino-N-[2-(1-oxy-piperidin-1-yl)ethyl]nicotinamide
in the form of the base or of an addition salt with an acid or in the form of a hydrate or a solvate.

16. A compound of formula: 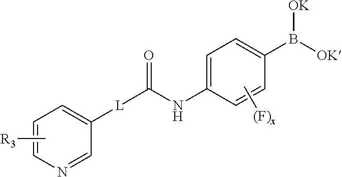
in which L represents a —CH═CH— or —(CH₂)ₙ—NH— group in which the NH group is attached to the C═O, R₃ is in the 5 and/or 6 position on the pyridine nucleus and represents at least one substituent of the pyridine nucleus chosen from a hydrogen atom, a fluorine atom, a (C₁-C₄)alkyl group or an —NR_{c}R_{d} group in which R_{c} and R_{d} represent a hydrogen atom or a (C₁-C₄)alkyl group and K and K′ represent a hydrogen atom, an alkyl group or an aryl group which are optionally connected to one another to form, together with the boron atom and the two oxygen atoms, a 5- to 7-membered ring optionally substituted by at least one (C₁-C₄)alkyl group or to which is optionally fused, over two consecutive carbon atoms on the said ring, a phenyl group.

17. The compound according to claim 13, wherein —B(OK)(OK′) represents one of the following groups:

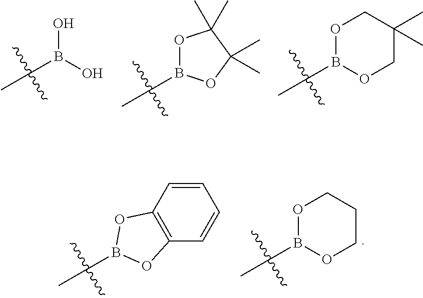

18. A compound of formula 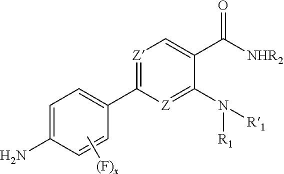
or of formula 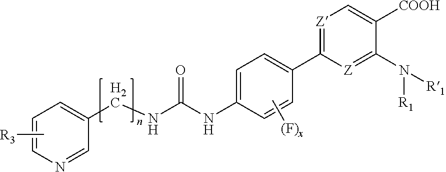
in which R₁ represents a hydrogen atom or a (C₁-C₆)alkyl, (C₃-C₆)cycloalkyl or phenyl group, R′₁ represents a hydrogen atom or a (C₁-C₆)alkyl group, R₂ represents:
a (C₃-C₆)cycloalkyl group;
a (C₁-C₆)alkyl group, optionally substituted by:
one or more hydroxyl or (C₁-C₄)alkoxy groups;
an —NRₐR_{b} group in which Rₐ and R_{b} represent, independently of one another, a hydrogen atom or a (C₁-C₆)alkyl group or form, together with the nitrogen atom to which they are connected, a (C₄-C₆)heterocycloalkyl group optionally comprising, in the ring, the —S(O)_{q}— group with q=0, 1 or 2 or the —NH— or —N(C₁-C₄alkyl)- group and being optionally substituted by one or more substituent(s), which are identical to or different from one another when there are several of them, chosen from an
—OH, (C₁-C₄)alkoxy or (C₁-C₄)alkyl group,
R₃ represents at least one substituent of the pyridine nucleus chosen from a hydrogen atom, a fluorine atom, a (C₁-C₄)alkyl group or an —NR_{c}R_{d} group in which R_{c} and R_{d} represent a hydrogen atom or a (C₁-C₄)alkyl group, Z represents N or CH, Z′ represents N or CH and x is an integer having the value 1 or 2, representing the number of fluorine atom(s) attached to the central phenyl nucleus.

19. A pharmaceutical composition comprising the compound according to claim 1.

20. The pharmaceutical composition of claim 19 further comprising at least one pharmaceutically acceptable excipient.

21. (canceled)

22. A method of treating or preventing cancer in a patient in need thereof comprising administering to said patient a therapeutically effective amount of the pharmaceutical composition of claim 19.

23. A compound chosen from the following list: [chemical structure]
